(19) United States
(12) Patent Application Publication
(10) Pub. No.: US 2009/0068672 A1
(43) Pub. Date: Mar. 12, 2009

(54) DETECTION SYSTEM

(75) Inventors: Martin Alan Lee, Wiltshire (GB); Tom Brown, Southampton (GB); Diane Rachel Sutton, Wiltshire (GB); Mark Andrew Laverick, Wiltshire (GB)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: ENIGMA DIAGNOSTICS LIMITED, WILTSHIRE (GB)

(21) Appl. No.: 12/279,088

(22) PCT Filed: Feb. 16, 2007
(86) PCT No.: PCT/GB2007/000559

§ 371 Date: Sep. 30, 2008

(30) Foreign Application Priority Data
Feb. 16, 2006 (GB) 0603190.0

(51) Int. Cl. C12Q 1/68

(52) U.S. Cl. 435/6

(57) ABSTRACT

The use of a red nucleic acid stain, in particular red fluorescent SYTO® dye in various methods used for the detection or characterisation of nucleic acids is described. In particular, the red nucleic acid stains have been found to be particularly compatible with the polymerase chain reaction (PCR), and therefore form the basis of enhanced detection methods.

The present invention provides the use of reagents in methods for detecting or characterising nucleic acids, methods for detecting a target polynucleotide in a sample, for example by quantitatively monitoring an amplification reaction, as well as to kits for use in these methods. The method is particularly suitable for the detection of polymorphisms or allelic variation and so may be used in diagnostic methods

Known fluorescence polymerase chain reaction (PCR) monitoring techniques include both strand specific and generic DNA intercalator techniques that can be used on a few second-generation PCR thermal cycling devices. These reactions are carried out homogeneously in a closed tube format on thermal cyclers. Reactions are monitored using a fluorimeter. The precise form of the assays varies but often relies on fluorescence energy transfer or FET between two fluorescent moieties within the system in order to generate a signal indicative of the presence of the product of amplification.

Generic methods utilise DNA intercalating dyes that exhibit increased fluorescence when bound to double stranded DNA species. Fluorescence increase due to a rise in the bulk concentration of DNA during amplifications can be used to measure reaction progress and to determine the target molecule copy number. Furthermore, by monitoring fluorescence with a controlled change of temperature, DNA melting curves can be generated, for example, at the end of PCR thermal cycling.

When generic DNA methods are used to monitor the rise in bulk concentration of nucleic acids, these processes can be monitored with a minimal time penalty (compared to some other known assays discussed below). A single fluorescent reading can be taken at the same point in every reaction. End point melting curve analysis can be used to discriminate artefacts from amplicon, and to discriminate amplicons. Melting peaks of products can be determined for concentrations that cannot be visualised by agarose gel electrophoresis.

In order to obtain high resolution melting data, for example for multiple samples, the melt experiment must be performed slowly on existing hardware taking up to five minutes. However, by continually monitoring fluorescence amplification, a 3D image of the hysteresis of melting and hybridisation can be produced. This 3D image is amplicon dependent and may provide enough information for product discrimination.

It has been found that DNA melting curve analysis in general is a powerful tool in optimising PCR thermal cycling. By determining the melting temperatures of the amplicons, it is possible to lower the denaturing temperatures in later PCR cycles to this temperature. Optimisation for amplification from first generation reaction products rather than the target DNA, reduces artefact formation occurring in later cycles. Melting temperatures of primer oligonucleotides and their complements can be used to determine their annealing temperatures, reducing the need for empirical optimisation.

The generic intercalator methods however are only quasi-strand-specific and therefore is not very useful where strand specific detection is required.

Strand specific methods utilise additional nucleic acid reaction components to monitor the progress of amplification reactions. These methods often use fluorescence energy transfer (FET) as the basis of detection. One or more nucleic acid probes are labelled with fluorescent molecules, one of which is able to act as an energy donor and the other of which is an energy acceptor molecule. These are sometimes known as a reporter molecule and a quencher molecule respectively. The donor molecule is excited with a specific wavelength of light which falls within its excitation spectrum and subsequently it will emit light within its fluorescence emission wavelength. The acceptor molecule is also excited at this wavelength by accepting energy from the donor molecule by a variety of distance-dependent energy transfer mechanisms. A specific example of fluorescence energy transfer which can occur is Fluorescence Resonance Energy Transfer or “FRET”. Generally, the acceptor molecule accepts the emission energy of the donor molecule when they are in close proximity (e.g. on the same, or a neighbouring molecule). The basis of fluorescence energy transfer detection is to monitor the changes at donor and acceptor emission wavelengths.

There are two commonly used types of FET or FRET probes, those using hydrolysis of nucleic acid probes to separate donor from acceptor, and those using hybridisation to alter the spatial relationship of donor and acceptor molecules.

Hydrolysis probes are commercially available as TaqMan™ probes. These consist of DNA oligonucleotides that are labelled with donor and acceptor molecules. The probes are designed to bind to a specific region on one strand of a PCR product. Following annealing of the PCR primer to this strand, Taq enzyme extends the DNA with 5′ to 3′ polymerase activity. Taq enzyme also exhibits 5′ to 3′ exonuclease activity. TaqMan™ probes are protected at the 3′ end by phosphorylation to prevent them from priming Taq extension. If the TaqMan™ probe is hybridised to the product strand, an extending Taq molecule may also hydrolyse the probe, liberating the donor from acceptor as the basis of detection. The signal in this instance is cumulative, the concentration of free donor and acceptor molecules increasing with each cycle of the amplification reaction.

The fact that signal generation is dependent upon the occurrence of probe hydrolysis reactions means that there is a time penalty associated with this method. Furthermore, the presence of the probe may interrupt the smooth operation of the PCR process.

In addition, it has been found that hydrolysis can become non-specific, particularly where large numbers of amplification cycles, for instance more than 50 cycles, are required. In these cases, non-specific hydrolysis of the probe will result in an unduly elevated signal.

This means that such techniques are not very compatible with rapid PCR methods which are becoming more prominent with the development of rapid hot air thermal cyclers such as the RapidCycler™ and LightCycler™ from Idaho Technologies Inc. Other rapid PCR devices are described for example in co-pending British Patent Application Nos. 9625442.0 and 9716052.7. The merits of rapid cycling over conventional thermal cycling have been reported elsewhere. Such techniques are particularly useful for example in detection systems for biological warfare where speed of result is important if loss of life or serious injury is to be avoided.

Furthermore, hydrolysis probes do not provide significant information with regard to hysteresis of melting since signal generation is, by and large, dependent upon hydrolysis of the probe rather than the melt temperature of the amplicon.

U.S. Pat. No. 5,491,063 describes a method for in-solution quenching of fluorescently labelled probes which relies on modification of the signal from a labelled single stranded oligonucleotide by a DNA binding agent. The difference in this signal which occurs as a result of a reduced chain length of the probe following probe cleavage (hydrolysis) during a polymerase chain reaction is suggested for providing a means for detecting the presence of a target nucleic acid.

Hybridisation probes are available in a number of forms. Molecular beacons are oligonucleotides that have complementary 5′ and 3′ sequences such that they form hairpin loops. Terminal fluorescent labels are in close proximity for FRET to occur when the hairpin structure is formed. Following hybridisation of molecular beacons to a complementary sequence the fluorescent labels are separated, so FRET does not occur, and this forms the basis of detection.

Pairs of labelled oligonucleotides may also be used. These hybridise in close proximity on a PCR product strand bringing donor and acceptor molecules together so that FRET can occur. Enhanced FRET is the basis of detection. Variants of this type include using a labelled amplification primer with a single adjacent probe.

The use of two probes, or a molecular beacon type of probe which includes two labelling molecules increases the cost involved in the process. In addition, this method requires the presence of a reasonably long known sequence so that two probes which are long enough to bind specifically in close proximity to each other are known. This can be a problem in some diagnostic applications, where the length of conserved sequences in an organism which can be used to design an effective probe, such as the HIV virus, may be relatively short.

Furthermore, the use of pairs of probes involves more complex experimental design. For example, a signal provided by the melt of a probe is a function of the melting-off of both probes. The study of small mismatches or where one of the probes is required to bind across a splice region (for example to detect RNA as compared to DNA in a sample where the sequence on either side of an intron can be utilised as the probe site) can yield incorrect results if the other probe melts first.

WO 99/28500 describes a very successful assay for detecting the presence of a target nucleic acid sequence in a sample. In this method, a DNA duplex binding agent and a probe specific for said target sequence, is added to the sample. The probe comprises a reactive molecule able to absorb fluorescence from or donate fluorescent energy to said DNA duplex binding agent. This mixture is then subjected to an amplification reaction in which target nucleic acid is amplified, and conditions are induced either during or after the amplification process in which the probe hybridises to the target sequence. Fluorescence from said sample is monitored.

As the probe hybridises to the target sequence, a DNA duplex binding agent such as an intercalating dye is trapped between the strands. In general, this would increase the fluorescence at the wavelength associated with the dye. However, where the reactive molecule is able to absorb fluorescence from the dye (i.e. it is an acceptor molecule), it accepts emission energy from the dye by means of FET, especially FRET, and so it emits fluorescence at its characteristic wavelength. Increase in fluorescence from the acceptor molecule, which is of a different wavelength to that of the dye, will indicate binding of the probe in duplex form.

Similarly, where the reactive molecule is able to donate fluorescence to the dye (i.e. it is a donor molecule), the emission from the donor molecule is reduced as a result of FRET and this reduction may be detected. Fluorescence of the dye is increased more than would be expected under these circumstances.

The signal from the reactive molecule on the probe is a strand specific signal, indicative of the presence of target within the sample. Thus the signal changes in fluorescence from the reactive molecule, which are indicative of the formation or destabilisation of duplexes involving the probe, are preferably monitored.

DNA duplex binding agents, which may be used in the process, are any entity which adheres or associates itself with DNA in duplex form and which is capable of acting as an energy donor or acceptor. Particular examples are intercalating dyes as are well known in the art.

The use of a DNA duplex binding agent such as an intercalating dye and a probe which is singly labelled is advantageous in that these components are much more economical than other assays in which doubly labelled probes are required. By using only one probe, the length of known sequence necessary to form the basis of the probe can be relatively short and therefore the method can be used, even in difficult diagnostic situations. The assay in this case is known as ResonSense®.

The DNA duplex binding agent used in the ResonSense® assay is typically an intercalating dye, for example SYBR Green such as SYBR Green I, SYBR Gold, ethidium bromide and YOPRO-1, which are themselves fluorescent.

In order for FET, such as FRET, to occur between the reactive molecule and the dye, the fluorescent emission of the donor (which may either be the intercalating dye or the reactive molecule on the probe) must be of a shorter wavelength than the acceptor (i.e. the other of the dye or the reactive molecule). The fluorescent signals produced by the molecules used as donor and/or acceptor can be represented as peaks within the visible spectrum. A particular known embodiment of ResonSense® utilises a universal donor system where light (˜470 nm) is used to excite the DNA binding agent SYBR® Gold or SYBR® Green-1. Energy is transferred to particular cyanine dyes such as Cy5 and Cy 5.5.

Generally, there will be at least some overlap in the wavelengths of the emission. Even where the signals are sharp peaks, there will be some “leakage” of signal from fluorescent molecules so that it is generally necessary to resolve the strand specific peak produced by the probe from the DNA duplex binding agent signal. This can be done, for example by determining empirically the relationship between the spectra of the donor and acceptor and using this relationship to normalise the signals from the donor and acceptor. SYBR dyes have a particularly broad spectrum of emission, and therefore a colour deconvolution algorithm is necessary for application. They are generally green in nature.

Additionally, SYBR dyes can become limiting in the reaction such that in multiplex reactions probe signal may diminish with increased amplification such that one probe signal may out compete others.

However, the SYBR dyes are widely used in various applications including nucleic acid detection and melting point analysis, largely because their fluorescent properties “match” those of other commonly utilised fluorophores such as Fluoroscein, and this allows the same optics (blue diode/˜520 nM filter) to be used in their detection.

The use of specifically Sybr Green and a related dye, pico green, in real-time PCT is described in U.S. Pat. No. 5,569,627 and EP-B-1179600.

Although SYBR Green is widely used in real-time PCR, in order to use this dye effectively, it is generally necessary to make careful optimisation of the conditions. This may require the inclusion of specific reagents such as DMSO, bovine serum albumin and Triton X-100. Inhibition of the PCR itself in a concentration dependent manner is also observed when SYBR green is included and this frequently necessitates the addition of magnesium chloride.

The use of a different dye, SYTO 9, a green dye as an alternative to SYBR Green has been discussed by Monis et al. Analytical Biochemistry 340 (2005) 24-34.

WO2004/033726 describes a variation of the ResonSense® method in which a DNA duplex binding agent which can absorb fluorescent energy from the fluorescent label on the probe but which does not emit visible light, so as to interfere with the signal is used. WO02/097132 describes a further variation in which a particular probe type is utilised.

However, the applicants have found a particularly advantageous combination for use in methods of this type.

According to the present invention there is provided the use of a nucleic acid stain, and in particular a red nucleic acid stain, in the detection of nucleic acids in a PCR reaction, in particular in a real-time PCR reaction. In this context, PCR reactions include reverse-transcriptase PCR (RT-PCR) as well as DNA amplification reactions.

As used herein the expression “nucleic acid stain” refers to products and compounds which are used or are proposed to be preferentially used for staining of cells or their contents. They exclude dyes such as SYBR®green or SYBR® gold as well as ethidium bromide.

In particular, nucleic acid stains used are those which include or are derived from a thiazole orange moiety of general formula (A)

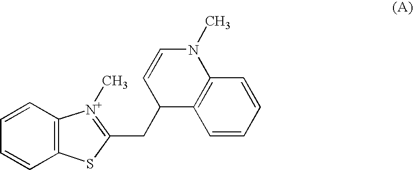

Red nucleic acid stains generally emit fluorescence at wavelengths in excess of 600 nm, for example from 610-690 nm. They may be cell permeant, such as the SYTO® Red Fluorescent nucleic acid stains available from Molecular Probes, which are known and recommended for use in many biological investigations where they enter cells and stain particularly cell nuclei. As a result they may show intranuclear bodies, as well as mitochondria. Red SYTO dyes have never been utilised previously in relation to the detection of nucleic acids in vitro, for example in the context of an amplification reaction such as a polymerase chain reaction (PCR).

Stains of this type are generally cyanine dyes for example as described in WO94/024213, WO96/013552, WO00/066664, WO02/028841, WO04/025259, wo05/038460, WO05/047242, wo05/047901, WO05/056687 and WO05/064336 and in particular WO 00/66664 the content of which are incorporated herein by reference. In particular, the stains are cyanine dyes as described generally in U.S. Pat. No. 5,658,751 which are red in colour.

These dyes generally comprise an asymmetrical chemical structure comprising two different heterocyclic ring systems which may be optionally substituted, which are linked by a bridging methine group of sub-formula (i)

—(CR³²═CR³¹)ₙ—CR³⁰═ (i)

where n is 0, 1 or 2, R³⁰, R³¹ and R³² are independently selected from hydrogen, C₁₋₆alkyl, C₃₋₁₀cylcloalkyl, aryl or heteroaryl. In particular at least one of R³⁰, R³¹ and R³² and preferably all are hydrogen.

As used herein, the term “aryl” refers to aromatic carbocyclic groups, for example phenyl or naphthyl. The term “heteroaryl” refers to aromatic cyclic groups, for example of from 5-20 atoms, at least one of which is a heteroatom selected from oxygen, nitrogen or sulphur. Heteroaryl groups are suitably mono or bicyclic in nature.

In general, in red stains useful in the present invention, n is 1, but other values of n may be acceptable, if the heterocyclic rings have the effect of shifting the emission to the red end of the spectrum.

For example, red stains may comprise compounds of when n is zero, and in these cases, they will generally include a modification in the ring structure, as compared to a green dye, which can lower the energy levels, for example by contributing electron density to the ring, such as a heteroatom, for example nitrogen.

Particular heterocyclic ring systems for attachment at either side of the methane group of sub-formula (i) above are illustrated in WO94/024213, WO96/013552, WO00/066664, WO02/028841, WO04/025259, WO05/038460, WO05/047242, wo05/047901, WO05/056687 and WO05/064336 and in particular WO 00/66664.

In particular, the compounds described in these references which include the basic benzothiazolyl and quinolinium ring may be prepared.

Thus for example, nucleic acid stains may have a first heterocyclic ring that is a substituted aza-benzolium ring, linked to a second heterocyclic ring system that is a pyridine, a quinoline, a pyridinium or a quinolinium group, by way of a methine linker of sub-formula (i) above.

In particular, such compounds may fall within the general formula (I):

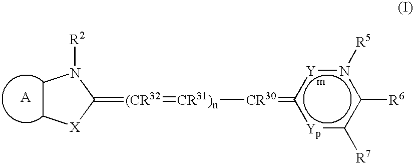

wherein A forms one or two fused aromatic rings having six atoms in each ring, at least one of which is optionally a nitrogen atom, said ring or rings being optionally further substituted one or more times by alkyl having from 1-6 carbons, alkoxy having from 1-6 carbons, trifluoromethyl, halogen, or -L-R; or -L-S;
X is 0, S, Se, NR¹⁵, or CR¹⁶R¹⁷, where R¹⁵ is H or an alkyl group having 1-6 carbons; and R¹⁵ and R¹⁷, which may be the same or different, are independently alkyl groups having 1-6 carbons, or
R³⁶ and R¹⁷ taken in combination complete a five or six membered saturated ring; a is 0 or 1;
R² is hydrogen, an alkyl group having 1-6 carbons that is optionally substituted by sulphonate, carboxy, or amino; or R² is -L-R. or -L-S; or TAIL; or BRIDGE-DYE;
n=0, 1 or 2, and preferably is 1;

### Y is —CR³═CR⁴—;

p and m=0 or 1, such that p+m=1;
R³, R⁴, R⁶, and R⁷ are independently H; an alkyl that is saturated or unsaturated, linear or branched, having 1-6 carbons; or a halogen; or a cyclic group (selected from an aryl, beteroaryl, or cycloalkyl having 3-10 carbons any of which may be optionally substituted by halogen, amino, alkyl, perfluoroalkyl, alkylamino, dialkylamino, alkoxy or carboxyalkyl, wherein each alkyl group has 1-6 carbons, or by a TAIL moiety); or —OR⁸, —SR⁸, —(NR⁸R⁹); or TAIL; or BRIDGE-DYE; or -L-Rₓ; or -L-S_{c}; where R⁸ and R⁹, which can be the same or different, are independently alkyl groups having 1-6 carbons; or 1-2 alicyclic or aromatic rings; or R⁸ and R⁹ taken in combination are —(CH₂)₂—V—(CH₂)₂— where V is a single bond, —O—, —CH₂—, or —NR¹⁰—, where R¹⁰ is H or an alkyl having 1-6 carbons;
or R⁶ and R⁷ form a fused aromatic ring —R¹¹=R¹²-R₁₃=R¹⁴— wherein R¹¹, R¹², R¹³, and R¹⁴ are optionally and independently alkyl. that are saturated or unsaturated, linear or branched, having 1-6 carbons; or —OR⁸, —SR⁸, or —(NR⁸R⁹); or a cyclic group (selected from an aryl, beteroaryl, or cycloalkyl having 3-10 carbons any of which may be optionally substituted by halogen, amino, alkyl, perfluoroalkyl, alkylamino, dialkylamino, alkoxy or carboxyalkyl, wherein each alkyl group has 1-6 carbons, or by a TAIL moiety)); or a TAIL; or BRIDGE-DYE; or -L-Rₓ or -L-S_{c};
R⁵ is an alkyl that is saturated or unsaturated, linear or branched, having 1-6 carbons; or R⁵ is a cyclic group (such as a carbocyclic or heterocyclic group of from 3 to 8 atoms); or R⁵ is TAIL; or BRIDGE-DYE; or -L-Rₓ; or -L-S_{c}; or R⁵ is a pair of electrons; R³⁰, R³¹, and R³² are independently H, alkyl having 1-6 carbons, cycloalkyl having 3-10 carbons, aryl, or heteroaryl; wherein L and BRIDGE are independently a single covalent bond, or a covalent linkage that is linear or branched, cyclic or heterocyclic, saturated or unsaturated, having 1-16 nonhydrogen atoms selected from the group consisting of C, N, P, 0 and S, such that the linkage contains any combination of ether, thioether, amine, ester, amide bonds; or single, double, triple or aromatic carbon-carbon bonds; or phosphorus-oxygen, phosphorus-sulphur bonds, nitrogen-nitrogen or nitrogen-oxygen bonds; or aromatic or heteroaromatic bonds;
Rₓ is a reactive group;
S_{c} is a conjugated groups;
TAIL is a heteroatom-containing moiety;
DYE is a compound of the formula (IA)

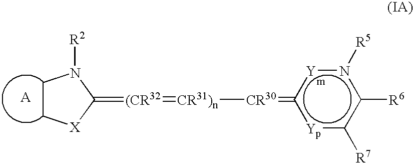

wherein A, X, R², n, Y, m, p, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R³⁰, R³¹, R³2, TAIL, or a cyclic group (selected from an aryl, beteroaryl, or cycloalkyl having 3-10 carbons any of which may be optionally substituted by halogen, amino, alkyl, perfluoroalkyl, alkylamino, dialkylamino, alkoxy or carboxyalkyl, wherein each alkyl group has 1-6 carbons, or by a TAIL moiety) are as defined above; that is bound to BRIDGE at one of R³, R⁴, R⁵, R⁶ or R⁷.

Particular examples of reactive groups Rₓ and conjugated groups S_{c} are as described in WO00/66664. In these cases, ring A contains a nitrogen atom.

Other examples of compounds of this general type are compounds which include the ring elements of thiazole orange of formula (A) above, and thus are compounds of formula (IB)

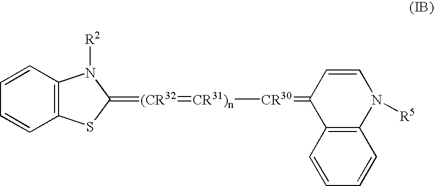

where n, R², R⁵, R³⁰, R³¹ and R³² are as defined above.

Such compounds may include substituents as described above for compounds of formula (IA). Thus a further series of compounds are compounds of formula (II) which are red;

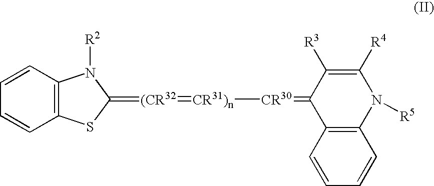

where R³, R⁴, R⁵, R³⁰, R³¹, R³² and n are as defined above, and R² is hydrogen, an alkyl group having 1-6 carbons that is optionally substituted by sulphonate, carboxy, or amino; provided the compounds are other than SYBR green and pico green. [The structures of SYBR green and pico green are represented as A and B respectively.]

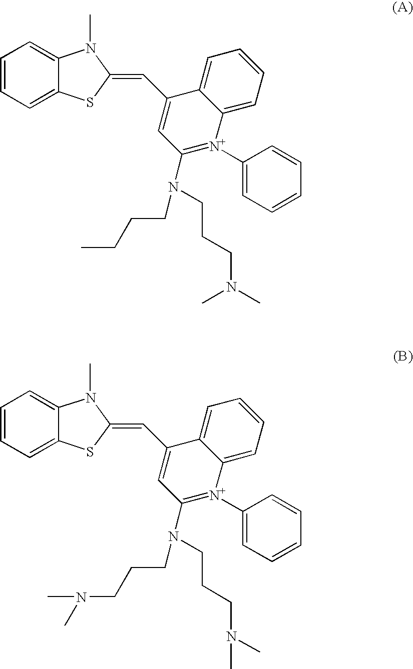

In this case, where n is 0, R³², R³¹, R³⁰ are all hydrogen, R³ is hydrogen and R⁴ is a TAIL group, R⁵ is suitably other than phenyl.

In particular R² is an alkyl group having from 1 to 6 carbon atoms such as methyl.

Furthermore, particular examples of n are 0 or 1, such as 0.

Where n is 1, R³² and R³³ are suitably hydrogen.

Particular examples of R³⁰ is hydrogen.

Suitably R⁵ is a TAIL group, or an aryl or heterocyclic group which is aromatic such as pyridyl. Compounds of formula (II) where R⁵ is a heteroaryl group form a particular embodiment of the invention, which is referred to as (IIA). In particular, when n is 0, R⁵ is a heteroaryl group such as pyridyl.

Particular examples of TAIL groups include groups of formula

LINK-SPACER-CAP

where LINK is the linking moiety by which TAIL is attached to the core structure of the dyes of the present invention. SPACER is a covalent linkage that connects LINK to CAP and CAP is the portion of TAIL that possesses a heteroatom component.

In particular, LINK is either a single covalent bond, an ether linkage (—O—), a thioether linkage (—S—), or an amine linkage (—NR²⁰—), where R²⁰ is selected from hydrogen, C₁₋₈alkyl group or a group

-SPACER′-CAP′

where SPACER′ and CAP′ are groups as defined below for SPACER and CAP respectively.

Suitably the LINK group is a group NR²⁰ where R²⁰ is as defined above.

Examples of SPACER groups include is a direct bond of a linear, branched, cyclic, heterocyclic, saturated or unsaturated arrangement of 1-16 C, N, P, O or S atoms. Alternatively, SPACER is a single covalent bond, provided that both LINK and SPACER are not simultaneously single covalent bonds. Preferably, the SPACER linkage must begin and end with a carbon atom. Typically, if SPACER consists of a single atom, it is required to be a carbon atom, so that the first and last atom in SPACER (in this specific instance, they are the same atom) is a carbon. The 1-16 atoms making up SPACER are combined using any appropriate combination of ether, thioether, amine, ester, or amide bonds; or single, double, triple or aromatic carbon-carbon bonds; or phosphorus-oxygen bonds; or phosphorus-sulphur bonds; or nitrogen-nitrogen bonds; or nitrogen-oxygen bonds; or aromatic or heteroaromatic bonds. SPACER is further substituted by hydrogen to accommodate the valence state of each atom in SPACER.

Generally, the atoms of SPACER are arranged such that all heteroatoms in the linear backbone of SPACER are separated by at least one carbon atom, and preferably separated by at least two carbon atoms. Typically, SPACER is 1-6 carbon atoms in a linear or branched saturated chain. In one embodiment of the invention, SPACER incorporates a 6-membered aromatic ring (phenylene linkage). In another embodiment of the invention, SPACER incorporates a 5- or 6-membered heteroaromatic ring, wherein the heteroatoms are O, N, or S. Alternatively, SPACER incorporates amide linkages, ester linkages, simple ethers and thioethers, and amines in a linear arrangement, such as —CH₂CH₂(C═O)NHCH₂ CH₂ CH₂. Preferably, SPACER is an alkylene —(CH₂)ₖ—, where k=1−8), and in particular a propylene group.

LINK and SPACER, in combination, serve to attach a heteroatom-containing group, CAP, to the dye core structure. CAP may contain oxygen, sulphur or nitrogen, according to the formulas —OR²¹, —SR²¹, —NR²¹R²², or —N⁺R²¹R²²R²³.PSI.⁻ where R²¹, R²², and R²³ are independently H, or an optionally substituted linear or branched alkyl or cycloalkyl group having 1-8 carbons and PSI⁻ is a counterion which is suitably biologically compatible as described below.

Where any of R²¹, R²² and R²³ are alkyl or cycloalkyl, they are optionally substituted by one or more groups selected from halogen, hydroxy, alkoxy having 1-8 carbons, amino, carboxy, or phenyl, where phenyl is optionally further substituted by halogen, hydroxy, alkoxy having 1-8 carbons, amino, aminoalkyl having 1-8 carbons, or carboxyalkyl having 1-8 carbons.

In another embodiment of the invention, one or more of R²¹, R²² and R²³, taken in combination with SPACER forms a 5- or 6-membered ring that is aromatic, heteroaromatic, alicyclic or heteroalicyclic ring. When the 5- or 6-membered ring is heteroaromatic or heteroalicyclic, the ring contains 1-3 heteroatoms that are O, N or S. Alternatively, one or more of R²¹, R²², and R²³, taken in combination with R²⁰ and SPACER, forms a 5- or 6-membered ring that is aromatic, heteroaromatic, alicyclic or heteroalicyclic ring, as described above. Preferably, R²¹, R²² are hydrogen, or alkyls having 1-8 carbons. R²³ is typically H or alkyl having 1-8 carbons.

When CAP is —N⁺R²¹R²²R²³.PSI.⁻, the biologically compatible counterion PSI⁻ balances the positive charge present on the CAP nitrogen, which is a quaternary ammonium salt. As used herein, a substance that is biologically compatible is not toxic as used, and does not have a substantially deleterious effect on biomolecules. Examples of .PSI⁻ include, among others, chloride, bromide, iodide, sulphate, alkanesulphonate, arylsulphonate, phosphate, perchlorate, tetrafluoroborate, tetraarylboride, nitrate and anions of aromatic or aliphatic carboxylic acids. Preferred PSI⁻ counterions are chloride, iodide, perchlorate and various sulphonates.

Additionally, in some embodiments of the present invention, CAP incorporates a cyclic structure. In these embodiments, CAP typically incorporates a 5- or 6-membered nitrogen-containing ring, optionally including an additional heteroatom (typically oxygen), where the ring nitrogen is optionally substituted by R²³ to give an ammonium salt. Specific versions of CAP include, but are not limited to, those listed in Table 1 of U.S. Pat. No. 5,658,751, the content of which is incorporated herein by reference.

Particular examples of TAIL groups are —NR²⁰(C₁₋₈alkylene)NR²¹R²² where R²⁰, R²³ and R²² are as defined above. For instance, TAIL groups are groups of sub-formula (i), (ii), or (iii)

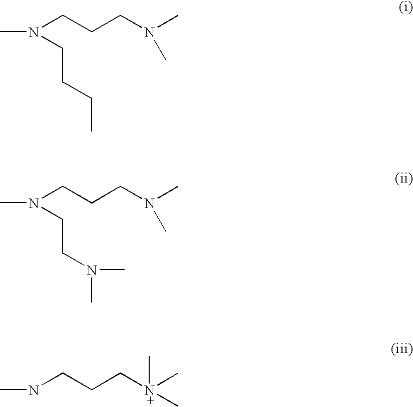

Suitably R³ is hydrogen and R⁴ is a TAIL group.

In a particular embodiment, the compound of formula (II) is a compound of formula (III)

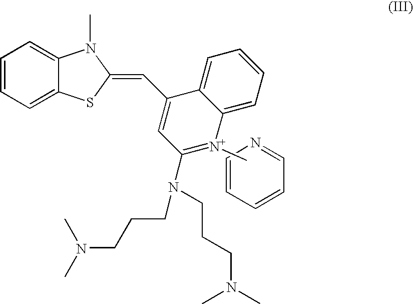

In one embodiment, the compound of formula (III) is a compound of formula (IIIA)

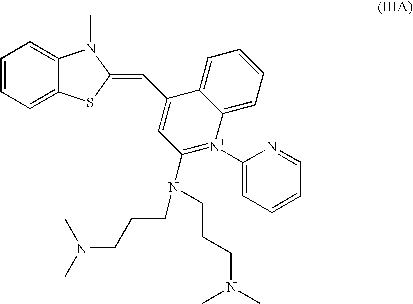

In another embodiment, the compound of formula (III) is a compound of formula (IIIB)

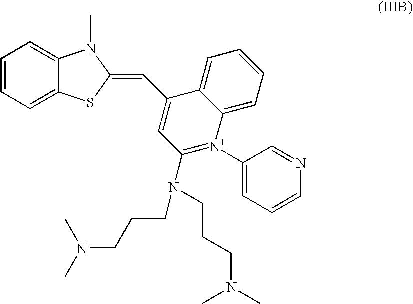

In yet another embodiment, the compound of formula (III) is a compound of formula (IIIC)

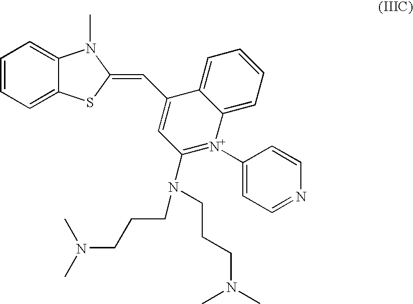

Particular examples of suitable red nucleic acid stains are the SYTO red nucleic acid stains available from Molecular Probes, such as SYTO® 17, SYTO® 59, SYTO® 60, SYTO® 61, SYTO® 62, SYTO® 63 and SYTO® 64. The spectral characteristics of these stains is illustrated in Table 1.

TABLE 1
Spectral characteristics of SYTO 17 and SYTO 59
to SYTO 64 red fluorescent nucleic acid stains.

| Dye | Cat# | Abs. (nm) | Em (nm) |
|---|---|---|---|
| SYTO 17 | S-7579 | 621 | 634 |
| SYTO 59 | S-11341 | 622 | 645 |
| SYTO 60 | S-11342 | 652 | 678 |
| SYTO 61 | S-11343 | 628 | 645 |
| SYTO 62 | S-11344 | 652 | 675 |
| SYTO 63 | S-11345 | 657 | 673 |
| SYTO 64 | S-11346 | 599 | 619 |

The applicants have found that these stains are particularly advantageous in the context of a real-time PCR detection method. It has been found that they do not inhibit the reaction, and can be added in a broad concentration range which is significantly less limiting than SYBR® dyes.

(Other members of the SYTO family including SYTO green nucleic acid stains may also have these benefits, but are less suitable for use in a reaction where their signal is not required to be measured.)

As a result, they may be utilised in monitoring the nucleic acid content and therefore the progress of the PCR, as well as in generic detection, the inclusion of internal controls, melt analysis etc.

They have been found to interact by FET or FRET with a wide variety of the most readily available dye molecules including fluorescein and the derivatives such as JOE.

These nucleic acid stains therefore provide a very advantageous addition to the sorts of fluorophores which may be utilised in amplification reactions and in particular in PCR amplification where monitoring of fluorescence is required.

Thus in a further aspect, the invention provides a method for detecting a nucleic acid sequence in a biological sample during amplification comprising the steps of:

adding a thermostable polymerase and primers configured for amplification of the target nucleic acid sequence to the biological sample:
amplifying the target nucleic acid sequence by the polymerase chain reaction in the presence of a nucleic acid stain as defined above and optionally additional signalling fluorophores, illuminating the biological sample with light at a wavelength absorbed by either the nucleic acid stain or the optional additional fluorophore; and
detecting a fluorescent emission from the sample related to the presence or amount of amplified target nucleic acid sequence in the sample.

The nucleic acid stain may be used alone to determine a reaction, in particular in real-time, using for example methods analogous to those described in U.S. Pat. No. 6,569,627, the content of which is incorporated herein by reference, since their lack of inhibition is useful in this context.

Generally however, they will be used in combination with another fluorophore, as the wavelength of emission of these stains is compatible with many of these.

In particular they may be utilised in real-time PCR reactions where the progress of the amplification is monitored. These reactions may include any of the real-time fluorescent assays described above including the TAQMAN™ assay, as well as assays which utilise dual hybridisation probes. In particular however, they are utilised in a ResonSense® assay, or in variations of this assay described in WO02/097132.

Thus in a particular embodiment, the invention provides a method for detecting the presence of a target nucleic acid sequence in a sample, said method comprising:

(a) adding to a sample suspected of containing said target nucleic acid sequence, a DNA duplex binding agent, and a probe specific for said target sequence, said probe comprising a reactive molecule able to absorb fluorescence from or donate fluorescent energy to said DNA duplex binding agent, wherein one of said reactive molecule or said DNA duplex binding agent is a nucleic acid stain as described above, and the other is a fluorophore, such as fluorescein or derivatives thereof,
(b) subjecting the thus formed mixture to an amplification reaction in which target nucleic acid is amplified,
(c) subjecting said sample to conditions under which the said probe hybridises to the target sequence, and
(d) monitoring fluorescence from said sample.

Suitably the nucleic acid stain is used as the DNA duplex binding agent.

By using a nucleic acid stain of the type described above, the problem with it supplying a signal that overlaps with that of the other signalling element of the system, which may be very many of the conventionally available fluorophores can be avoided or minimised. In particular, nucleic acid stains with a range of wavelengths are available, which means that it is possible to select appropriate combinations from among the known fluorophores, in particular reporter dyes, as well as excitation sources, to ensure that overlap of signal is minimised or does not occur. Thus the need to resolve the signals from the probe from the signal from the DNA duplex binding agent can be eliminated, and a broader bandwidth over which meaningful signal can be measured is available. This means that the apparatus, or at least the computational requirements placed upon the apparatus can be simplified.

As would be understood in the art, in order to monitor fluorescence, it is necessary to illuminate the sample at a wavelength of light which is absorbed by a fluorophore within the system, and then monitor emission of the fluorophore at the appropriate emission wavelength. More than one fluorophore may be monitored in this way, but illuminating the sample with more than one wavelength of light, and monitoring emissions at various wavelengths also. In a particular embodiment of the method, the sample is illuminated by light of a wavelength absorbed by the reactive molecule and the emission signal from the reactive molecule is monitored. There may be no need to monitor the signal from the nucleic acid stain, as this is unlikely to interfere with the signal from conventional fluorophores such as JOE and FAM.

The assay may therefore be carried out on a broader range of instruments.

Alternatively, any areas of free bandwidth in the visible spectrum may be exploited by incorporating additional probes, which include different labels which fluoresce at different wavelengths so that more that one target may be monitored at the same time. This may be particularly useful in the case of multiplex PCR reactions.

Nucleic acid stains as described above may be tested to see whether or not they absorb fluorescent energy for example, from a particular or from a range of conventional fluorophores using conventional methods. In particular, they may be included in a PCR reaction with a fluorescent agent, which may be a labelled probe or fluorescent intercalating agent to test the quenching properties. A suitable protocol for carrying out this testing is set out in Example 1 hereinafter.

The amount of nucleic acid stain which is added to the reaction mixture is suitably sufficient to cause measurable signal, for example quenching of the signal from the other fluorophore in the system, but not sufficient to inhibit amplification. The range of concentrations which will achieve this vary depending upon the precise nucleic acid stain being used, and can be determined by routine methods as illustrated hereinafter. Generally however, concentrations of the nucleic acid stain of from 1-10 μM, generally at about 5 μM.

The particular ResonSense® method is extremely versatile in its applications. It can be used to generate both quantitative and qualitative data regarding the target nucleic acid sequence in the sample, as discussed in WO2004/033726 for example. In particular, not only does the method provide for quantitative amplification, but also it can be used, additionally or alternatively, to obtain characterising data such as duplex destabilisation temperatures or melting points.

In the ResonSense® assay, the sample may be subjected to conditions under which the probe hybridises to the samples before, during or after the amplification reaction. The process therefore allows the detection to be effected in a homogenous manner, in that the amplification and monitoring can be carried out in a single container with all reagents added initially. No subsequent reagent addition steps are required. Neither is there any need to effect the method in the presence of solid supports (although this is an option).

The probe may comprise a nucleic acid molecule such as DNA or RNA, which will hybridise to the target nucleic acid sequence when the latter is in single stranded form. In this instance, step (c) will involve the use of conditions which render the target nucleic acid single stranded.

Probe may either be free in solution or immobilised on a solid support, for example to the surface of a bead such as a magnetic bead, useful in separating products, or the surface of a detector device, such as the waveguides of a surface plasmon resonance detector or a total internal reflection fluorescence detector. The selection will depend upon the nature of the particular assay being looked at and the particular detection means being employed.

In particular, the amplification reaction used will involve a step of subjecting the sample to conditions under which any of the target nucleic acid sequence present in the sample becomes single stranded. Such amplification reactions include the polymerase chain reaction (PCR) or the ligase chain reaction (LCR), but is preferably a PCR reaction.

It is possible then for the probe to hybridise during the course of the amplification reaction provided appropriate hybridisation conditions are encountered.

In a preferred embodiment, the probe may be designed such that these conditions are met during each cycle of the amplification reaction. Thus at some point during each cycle of the amplification reaction, the probe will hybridise to the target sequence, and whereupon the fluorescent signal will be quenched as a result of its close proximity to the DNA duplex binding agent trapped between the probe and the target sequence. As the amplification proceeds, the probe will be separated or melted from the target sequence and so the signal generated by it will be restored. Hence in each cycle of the amplification, a fluorescence peak from the fluorescent label at the point at which the probe is annealed is generated. The intensity of the peak will decrease as the amplification proceeds because more target sequence becomes available for binding to the probe.

By monitoring the fluorescence of the fluorescent label in the sample during each cycle, the progress of the amplification reaction can be monitored in various ways. For example, the data provided by melting peaks can be analysed, for example by calculating the area under the melting peaks and this data plotted against the number of cycles.

Fluorescence is suitably monitored using a known fluorimeter. The signals from these, for instance in the form of photo-multiplier current, are sent to a data processor board and converted into a spectrum associated with each sample tube. Multiple tubes, for example 96 tubes, can be assessed at the same time. Data may be collected in this way at frequent intervals, for example once every 10 ms, throughout the reaction.

This data provides the opportunity to quantitate the amount of target nucleic acid present in the sample.

In addition, the kinetics of probe hybridisation will allow the determination, in absolute terms, of the target sequence concentration. Changes in fluorescence from the sample can allow the rate of hybridisation of the probe to the sample to be calculated. An increase in the rate of hybridisation will relate to the amount of target sequence present in the sample. As the concentration of the target sequence increases as the amplification reaction proceeds, hybridisation of the probe will occur more rapidly. Thus this parameter also can be used as a basis for quantification. This mode of data processing useful in that it is not reliant on signal intensity to provide the information.

Suitable other fluorophores, including in particular fluorescent probe labels are rhodamine dyes or other dyes such as Cy5, Cy3, Cy5.5, fluorescein or derivatives thereof. Particular derivatives are carboxyfluorescein compounds sold under the trade name FAM or JOE, such as 5-carboxyfluorescein, 6-carboxyfluorescein, or their succinimidyl esters. As discussed above however, the precise selection of these will depend upon the nucleic acid stain utilised. However, by using in particular the red nucleic acid stains, the range of fluorophores available for use is extended.

Any labels may be attached to probes in a conventional manner. The position of the fluorescent label along the probe is immaterial although it general, they will be positioned at an end region of the probe.

Preferably they are positioned at the 3′ end of the probe, as they will then act as a steric or chemical blocking agent, to prevent extension of the probe by the polymerase during the amplification. This may avoid the need to take other measures, such as phosphorylation, in order to block the 3′ end of the probe during the amplification reaction.

It is possible to design the probe and the assay conditions such that the probe is hydrolysed by the DNA polymerase used in the amplification reaction, thereby releasing the fluorescent label. In this case, the probe will be designed to bind during the annealing and extension phase of the PCR reaction and the polymerase used in the assay will be one which has 5′-3′exonuclease activity. The released fluorescent label produces an increasing signal since it is no longer quenched by the DNA duplex binding agent. In this case therefore, the reaction can be monitored by observing the increasing signal of the free fluorescent label. The signal must be monitored at temperatures that are above those where the probe interacts with the target or product. In this case however, signal may be monitored during the annealing stage to determine the differential between the amounts of free and intact bound probe.

However, it is not necessary in this assay for the probe to be consumed in this way as signal production can be achieved without dissociating the probe.

In order to achieve a fully reversible signal which is directly related to the amount of amplification product present at each stage of the reaction, and/or where speed of reaction is of the greatest importance, for example in rapid PCR, it is preferable that the probe is designed such that it is released intact from the target sequence. This may be, for example, during the extension phase of the amplification reaction. However, since the signal is not dependent upon probe hydrolysis, the probe may be designed to hybridise and melt from the target sequence at any stage during the amplification cycle. For example probes which hybridise most strongly at a stage other than the extension phase of the cycle will ensure that interference with the amplification reaction is minimised.

Where probes which bind strongly at or below the extension temperature are used, their release intact from the target sequence can be achieved by using a 5′-3′ exonuclease lacking enzyme such as Stoffle fragment of Taq or Pwo, as the polymerase in the amplification reaction.

The probe may then take part again in the reaction, and so represents an economical application of probe.

The data generated in this way using probes which reversibly hybridise to the target and are not hydrolysed, can be interpreted in various ways. In its simplest form, a decrease in fluorescence of the fluorescent label at the probe annealing temperature in the course of or at the end of the amplification reaction is indicative of an increase in the amount of the target sequence present, suggestive of the fact that the amplification reaction has proceeded and therefore the target sequence was in fact present in the sample.

However, as outlined above, quantification is also possible by monitoring the amplification reaction throughout.

Finally, it is possible to obtain characterisation data and in particular melting point analysis, either as an end point measure or throughout, in order to obtain information about the sequence as will be discussed further below.

Thus, a preferred embodiment of the invention comprises a method for detecting nucleic acid amplification comprising:

performing nucleic acid amplification on a target polynucleotide in the presence of (a) a nucleic acid polymerase (b) at least one primer capable of hybridising to said target polynucleotide, (c) an oligonucleotide probe which is capable of binding to said target polynucleotide sequence and which contains a fluorescent label and (d) a nucleic acid stain, in particular a red nucleic acid stain as described above, which is capable of absorbing fluorescent energy from the said fluorescent label; and monitoring changes in fluorescence during the amplification reaction.

The amplification is suitably carried out using a pair of primers which are designed such that only the target nucleotide sequence within a DNA strand is amplified as is well understood in the art. The nucleic acid polymerase is suitably a thermostable polymerase such as Taq polymerase.

Suitable conditions under which the amplification reaction can be carried out are well known in the art. The optimum conditions may be variable in each case depending upon the particular amplicon involved, the nature of the primers used and the enzymes employed. The optimum conditions may be determined in each case by the skilled person. Typical denaturation temperatures are of the order of 95° C., typical annealing temperatures are of the order of 55° C. and extension temperatures are of the order of 72° C.

As before, in a particular embodiment, the sample is illuminated by light of a wavelength absorbed by the fluorescent label of the oligonucleotide probe and the emission signal from the fluorescent label is monitored in order to determine the progress of the reaction. Other fluorophores in the system may also be monitored if desired, for example in multiplex assays, and these may need to be resolved using conventional methods. However, there may be no need to monitor the signal from the nucleic acid stain provided this does not overlap or significantly interfere with the signal from the fluorescent label.

Suitably, the fluorescence is monitored throughout the amplification process, and preferably, at least at the same point during each amplification cycle. In particular, fluorescence needs to be monitored at the temperature at which the probe anneals to the target. For instance, this may be at a temperature of about 60° C.

As more target is formed, more probe becomes annealed to it, and is quenched as a result of it being brought into close proximity to the nucleic acid stain. This reduction in fluorescence indicates the progress of the amplification.

The polymerase such as Taq polymerase present in the sample will have the effect of removing the probe from the target. This effect occurs at a low level, at the sub-optimal temperature for the polymerase, such as the probe annealing temperature. Hence at this temperature, these two reactions, the binding of the probe at its annealing temperature and the effect of the polymerase to remove the probe from the target, will compete. Generally, the former reaction will dominate for a significant number of reaction cycles, allowing the amplification reaction to be monitored. Ultimately however, a rise in fluorescence may be observed, when the balance shifts and the effect of the polymerase becomes more dominant. Hence the results can reveal a “hook” effect, which is believed to occur when product re-annealing becomes more favourable than probe/product annealing, resulting in a change the direction of the fluorescence curve at the end of the amplification reaction. The data obtained using the method of the invention, can be processed to monitor the progress of the amplification reaction, and may therefore be used to quantify the amount of target present in the sample.

In order to interpret the data obtained, it may be necessary to make certain adjustments. For instance, in a conventional PCR monitoring reaction such as that described in WO 99/28500, the PCR reaction will lead to an exponential rise in fluorescence, and so baseline adjustments for background fluorescence will need to be derived from the lowest values obtained.

In contrast, in the method of the present application, the progress of a PCR reaction will lead to an exponential fall in fluorescence as progressively more of the labelled probe is quenched by the DNA duplex binding agent, and in particular the nucleic acid stain. Hence baseline adjustment needs to be based upon the highest levels of fluorescence achieved.

This is suitably done by taking the data from a sample reaction reaction and applying the following equations to every datapoint:

y=1/x

z=y−MIN

where x is the datapoint from the PCR machine, such as a LightCyler, Z is the baseline adjusted datapoint and MIN is the minimum value for y over the entire dataset. A plot of Z vs cycle number will allow appropriate baseline adjustments to be calculated.

The method can be used in hybridisation assays for determining characteristics of particular sequences.

Thus in a further aspect, the invention provides a method for determining a characteristic of a sequence, said method comprising;

a) adding to a sample suspected of containing said sequence, a fluorescently labelled probe specific for said target sequence and a DNA duplex binding agent able to absorb fluorescence from a fluorescent label on the probe, wherein one of the label on the probe or the DNA duplex binding agent is a nucleic acid stain, and in particular a red nucleic acid stain as described above,
(b) subjecting said sample to a variable set of reaction conditions during which the said probe hybridises to the target sequence,
(c) monitoring fluorescence from said sample and determining a particular reaction condition, characteristic of said sequence, at which fluorescence changes as a result of the hybridisation of the probe to the sample or destabilisation of the duplex formed between the probe and the target nucleic acid sequence.

Suitable reaction conditions include temperature, electrochemical, or the response to the presence of particular enzymes or chemicals. By monitoring changes in fluorescence as these properties are varied, information characteristic of the precise nature of the sequence can be determined. For example, in the case of temperature, the temperature at which the probe separates or “melts” from the target sequence can be determined. This can be extremely useful in for example, to detect and if desired also to quantitate, polymorphisms in sequences including allelic variation in genetic diagnosis. By “polymorphism” is included transitions, transversions, insertions, deletions or inversions which may occur in sequences, particularly in nature.

The hysteresis of melting of the probe will be different if the target sequence varies by only one base pair. Thus where a sample contains only a single allelic variant, the temperature of melting of the probe will be a particular value which will be different from that found in a sample which contains only another allelic variant. A sample containing both allelic variants which show two melting points corresponding to each of the allelic variants.

Similar considerations apply with respect to electrochemical properties, or in the presence of certain enzymes or chemicals. The probe may be immobilised on a solid surface across which an electrochemical potential may be applied. Target sequence will bind to or be repulsed from the probe at particular electrochemical values depending upon the precise nature of the sequence.

This embodiment can be effected in conjunction with amplification reactions such as the PCR reaction mentioned above, or it may be employed individually.

Further aspects of the invention include kits for use in the method of the invention. These kits will contain a nucleic acid stain, and in particular a red nucleic acid stain as described above. Other potential components of the kit include reagents used in amplification reactions such as DNA polymerase (including chemically modified TAQ for “hotstart” reactions), primers, buffers and adjuncts known to improve the PCR process such as the “hotstart” reagents such as antiTaq antibody, or pyrophosphate and a pyrophosphatase, as described in copending International Patent Application PCT/GB02/01861. The kit may additionally or alternatively include a probe for a target sequence which is fluorescently labelled. In particular, the nucleic acid stain is to absorb fluorescence from a fluorescent label on the probe.

The kits may include all the reagents together in a single container, or some may be in separate containers for mixing on site.

The use of nucleic acid stains as described above provides a Universal Acceptor arrangement where multiple light sources could be used to transfer energy to a single DNA binding dye. This gives rise to a number of advantages, including the fact that the assay should perform better in a multiplex. It may work on many platforms and it does not require the monitoring of the acceptor dye.

Furthermore, the range of dye wavelengths offers a new possibility. It could be possible to arrange to have both a Universal donor and a universal acceptor mechanism in the same reaction. A short wavelength (e.g. UV diode) could excite a label for energy transfer to a longer wavelength nucleic acid stain, used as a duplex binding agent as described above. A second diode could be used to excite the same nucleic acid stain for further transfer of energy to a second probe with a fluorescent label.

Most commercially available machines arranged to carry out PCR have multiple light sources, but SYBR dyes have become the industry standard. However, using another wavelength DNA binding dye that does not interfere with the probe label emission is a readily available technical option.

The invention will now be particularly described by way of example with reference to the accompanying diagrammatic drawings in which:

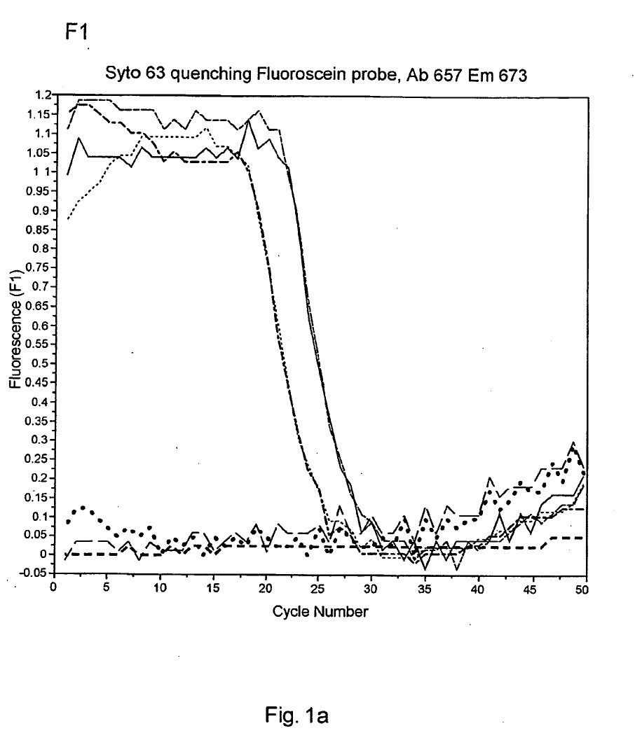

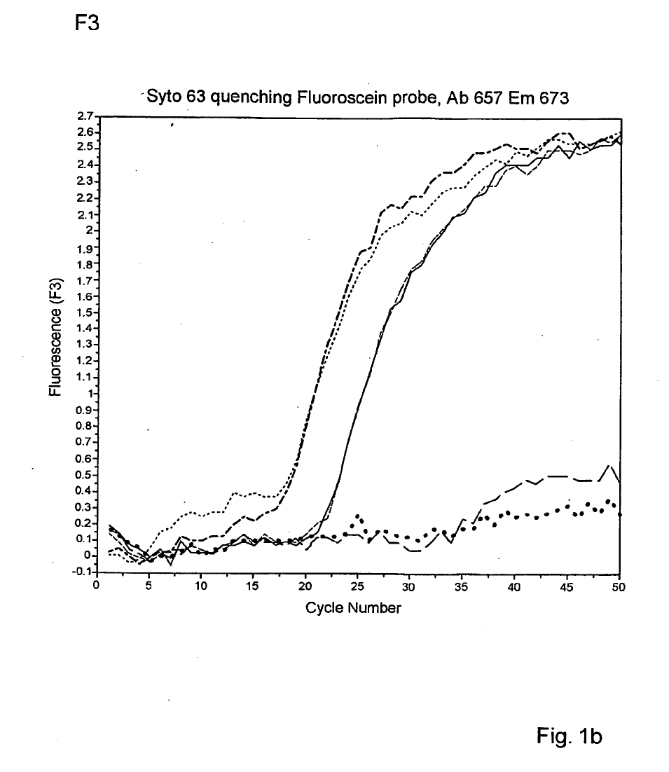

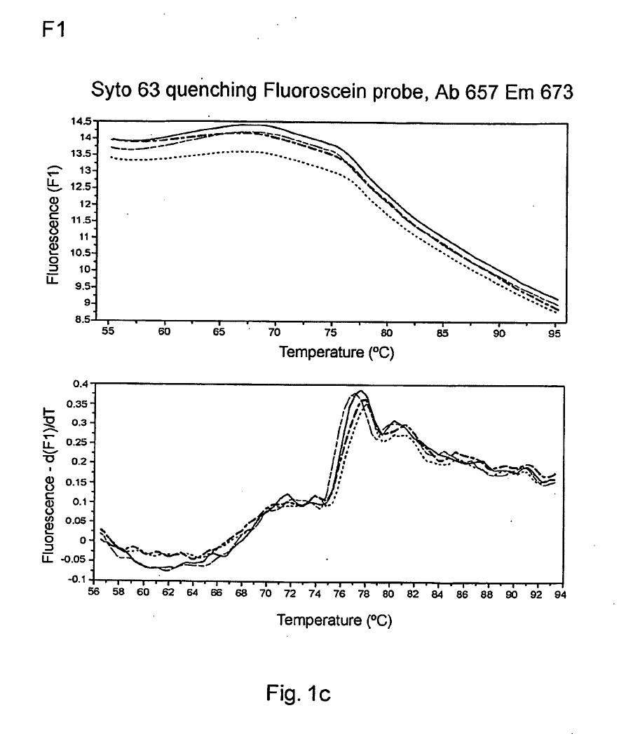

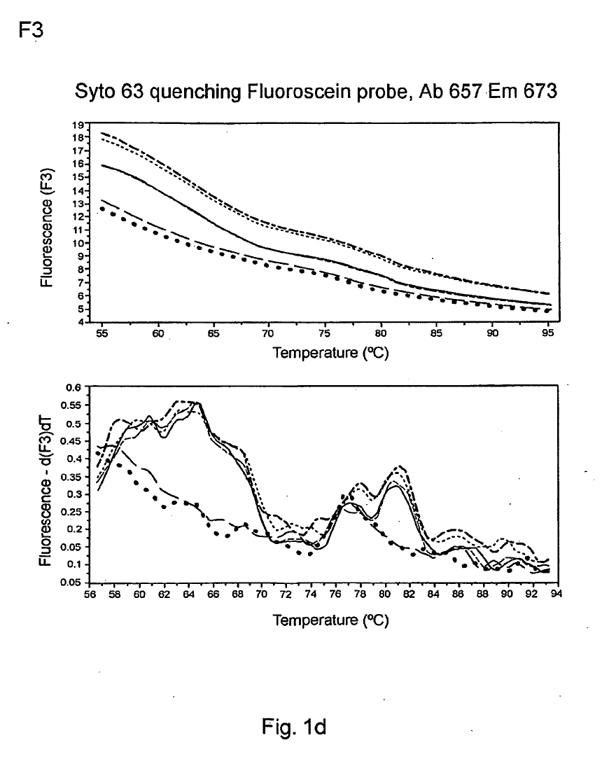

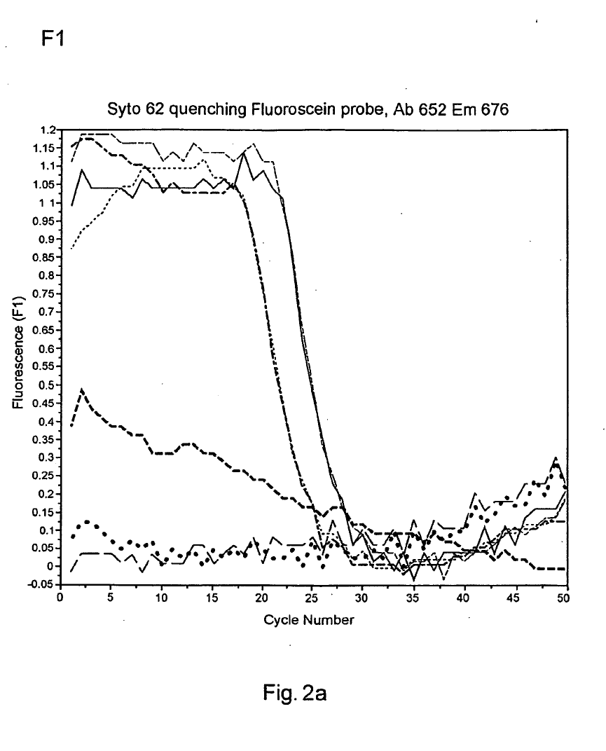

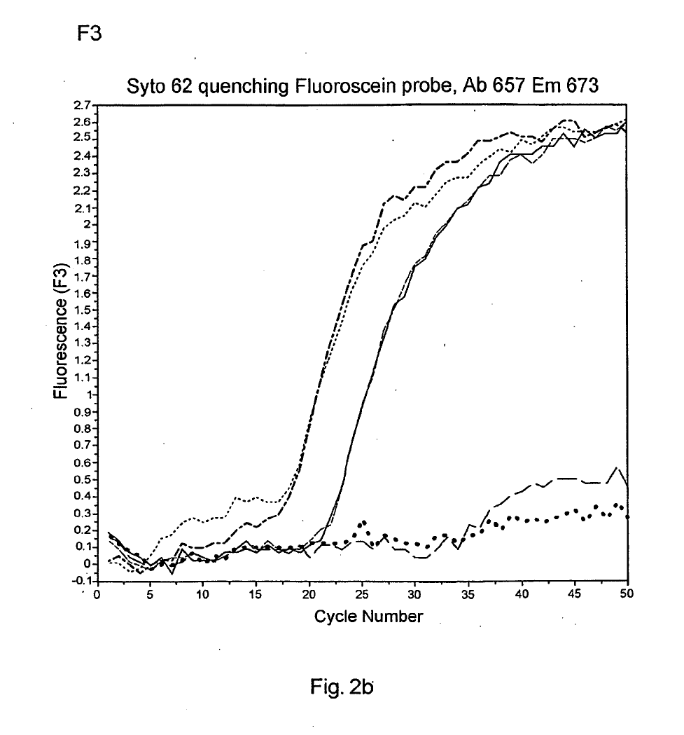

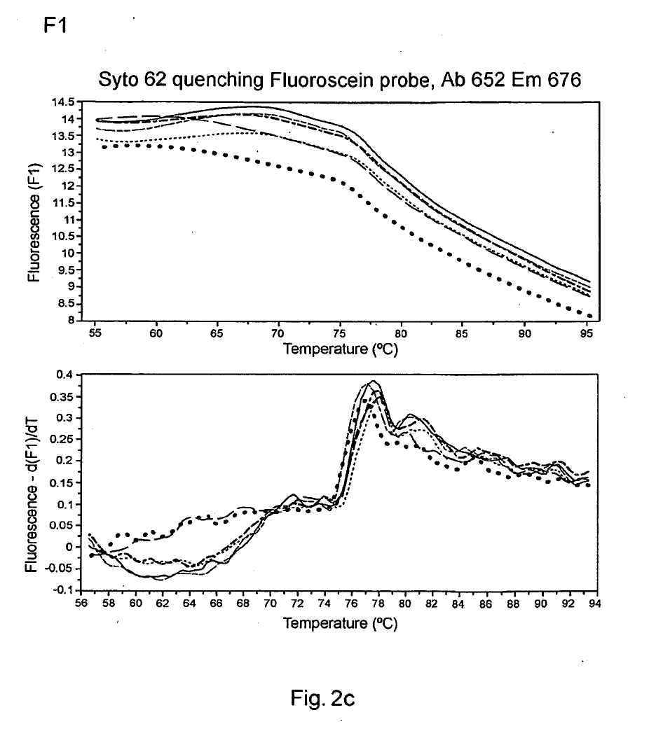

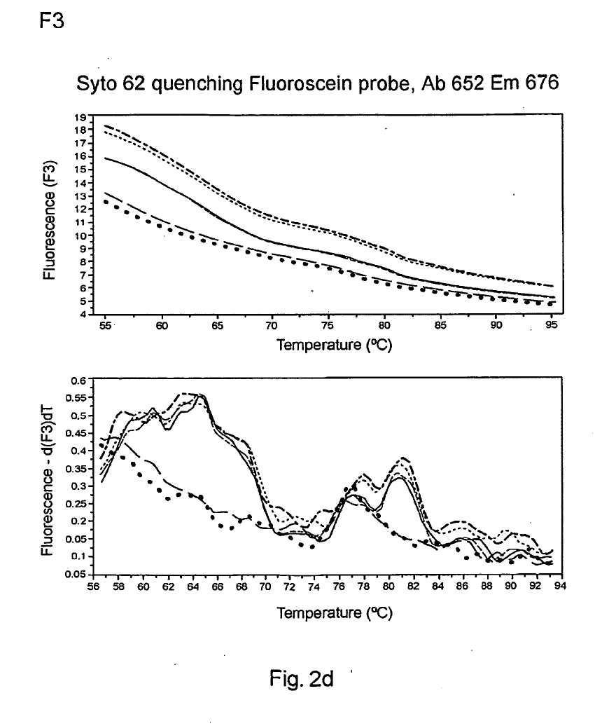

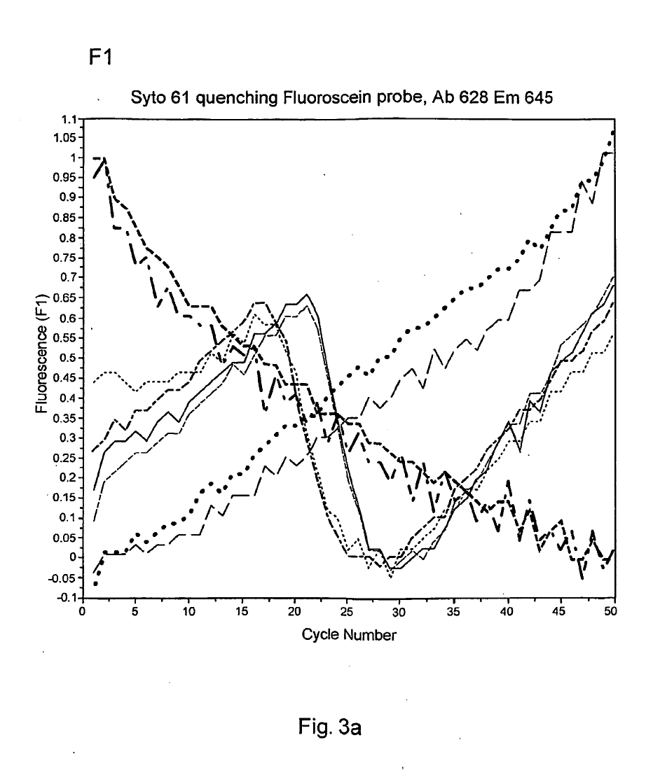

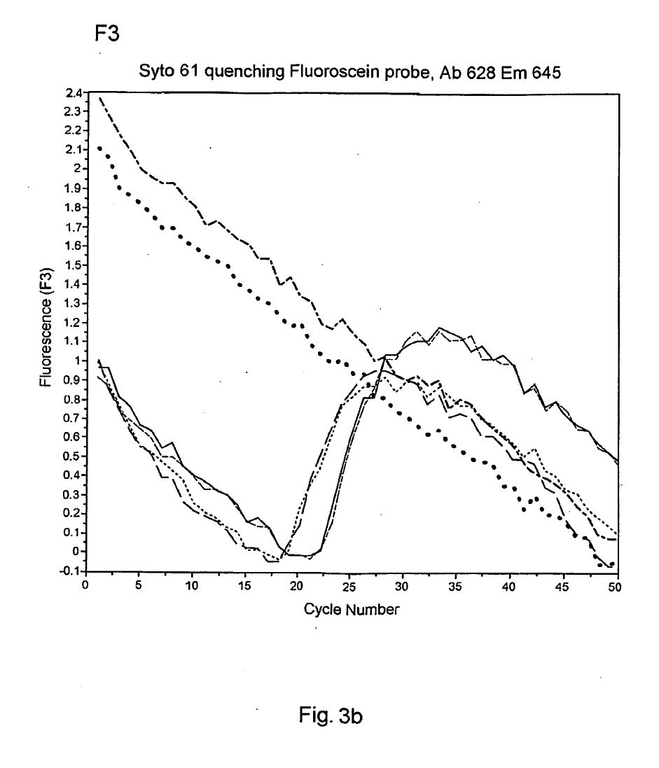

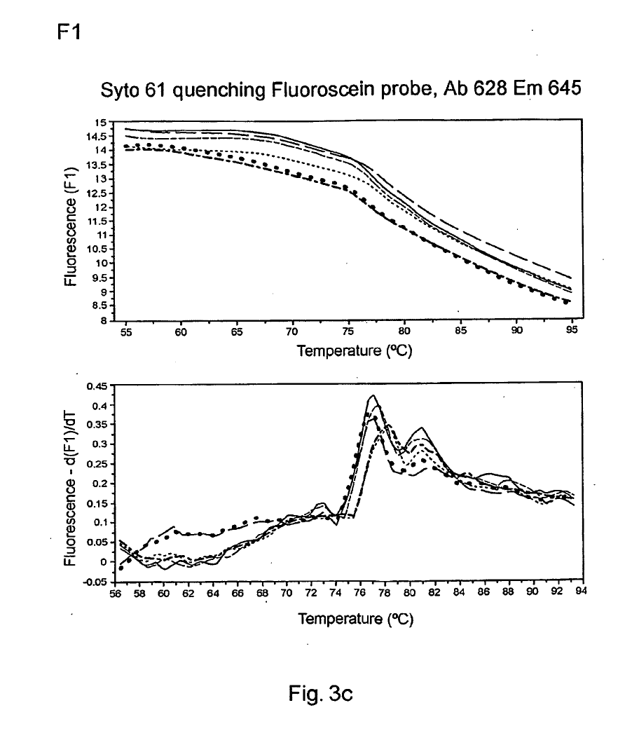

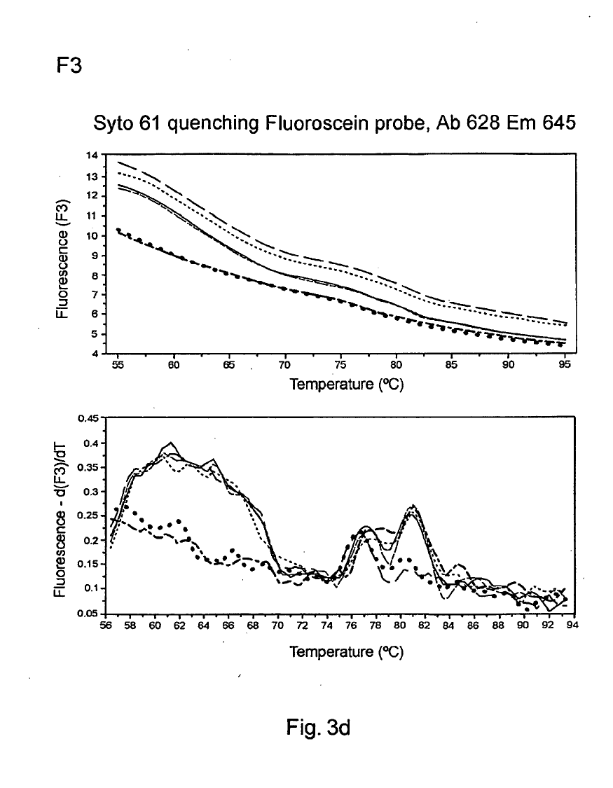

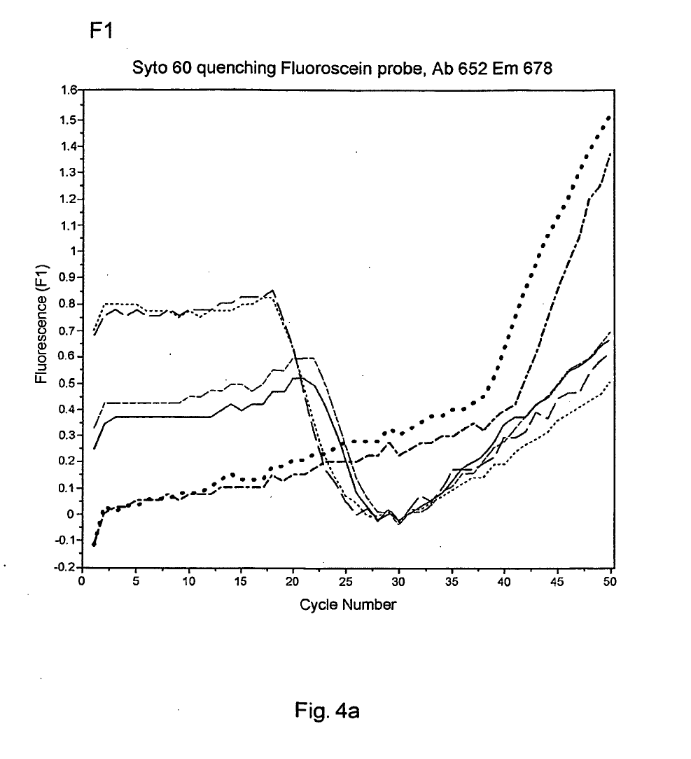

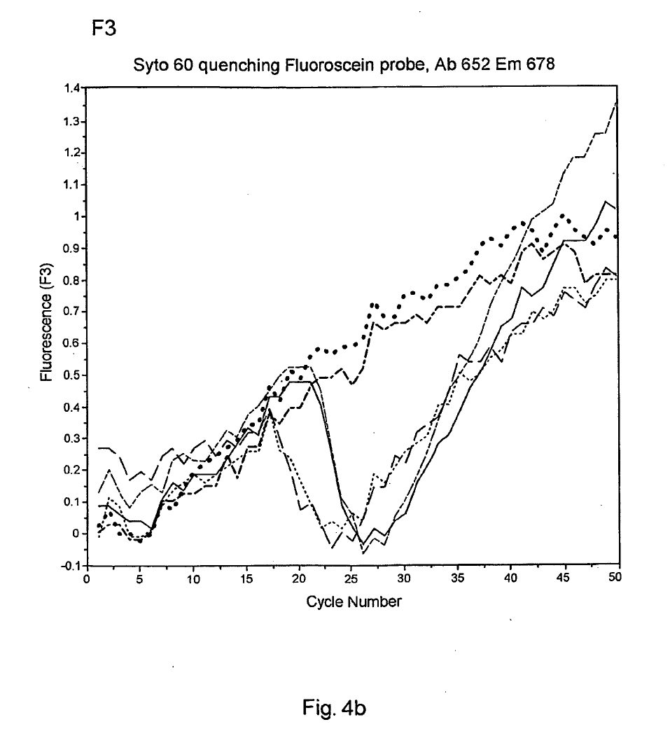

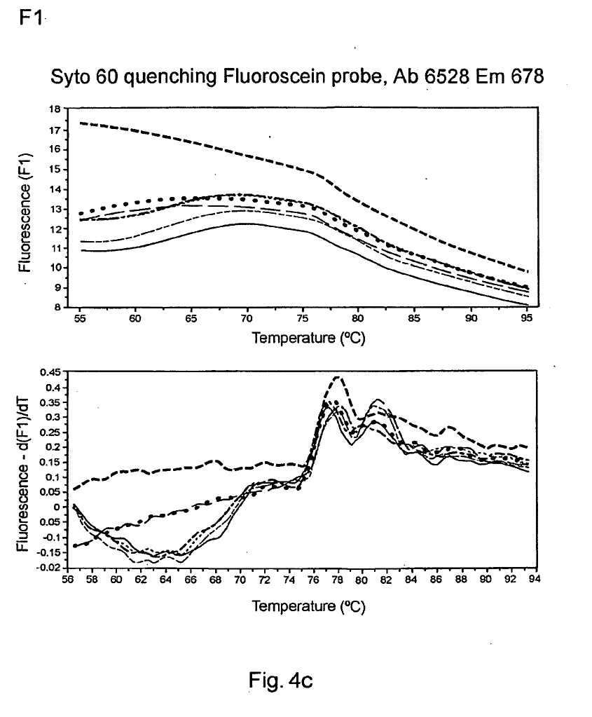

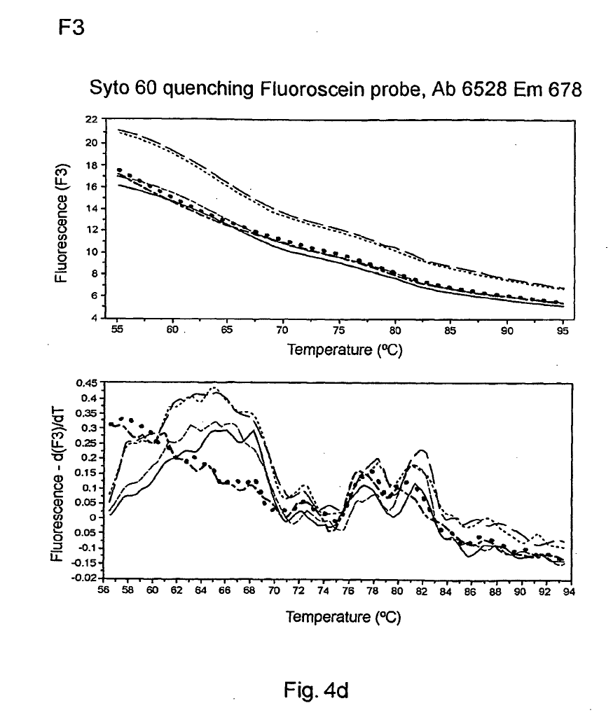

FIGS. 1-4 shows the results of the quenching of a fluorescein probe with SYTO 63, SYTO 62, SYTO 61 and SYTO 60 respectively;

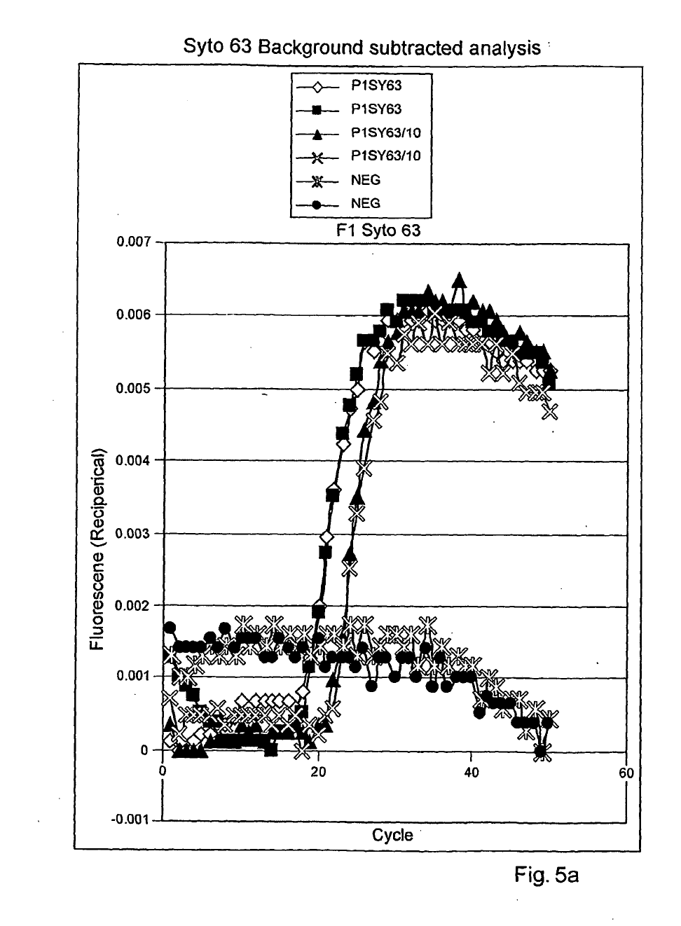

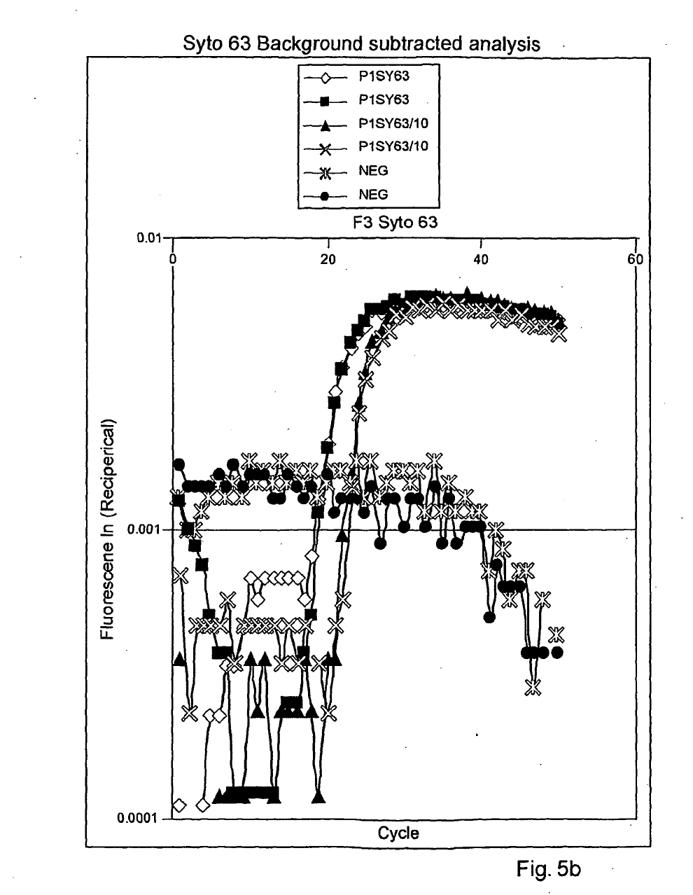

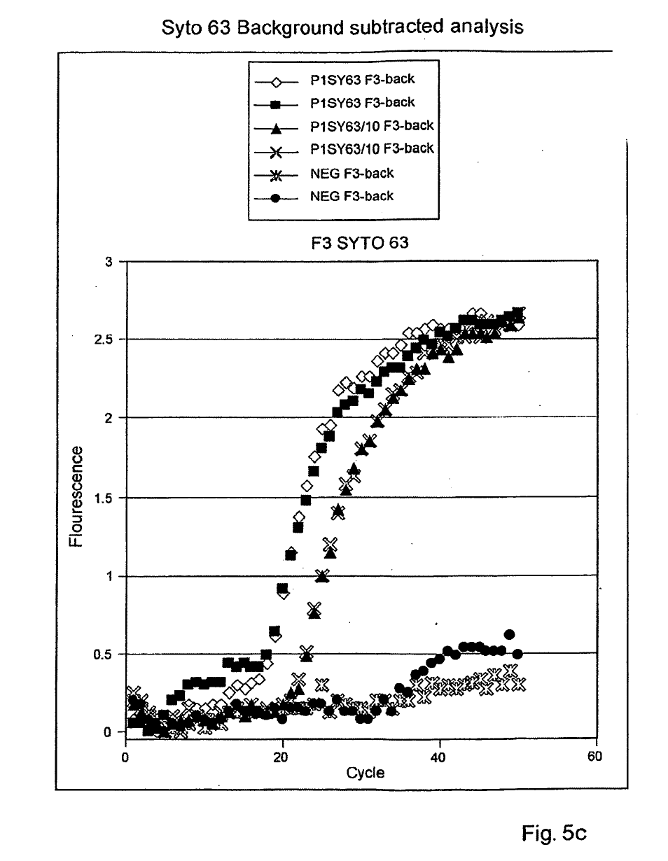

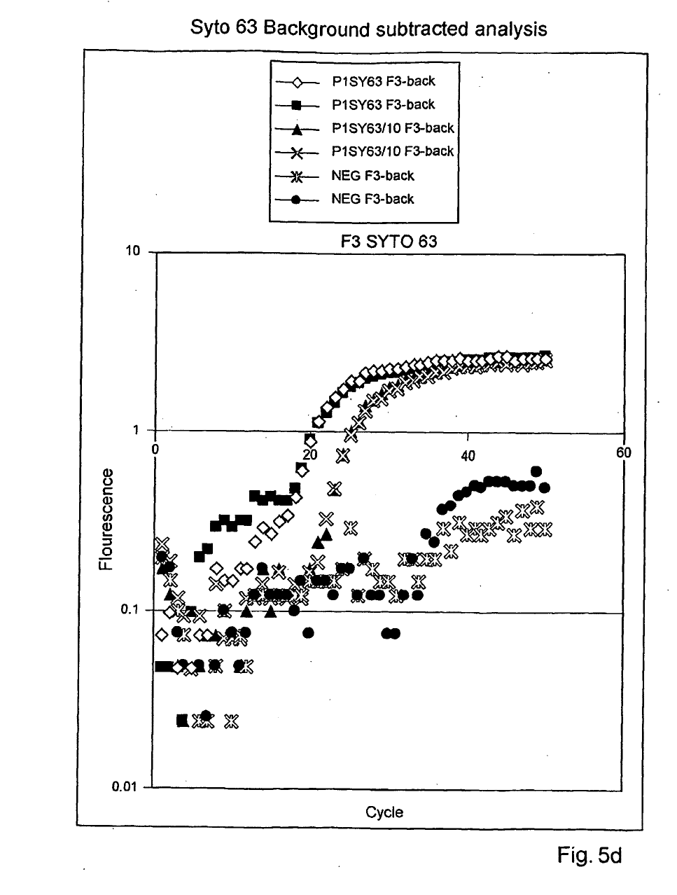

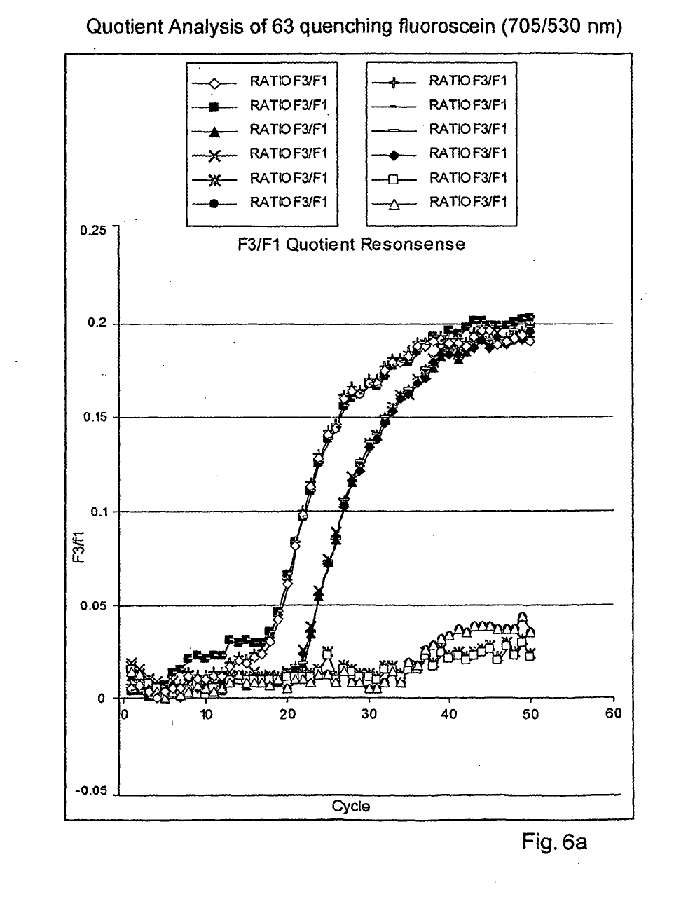

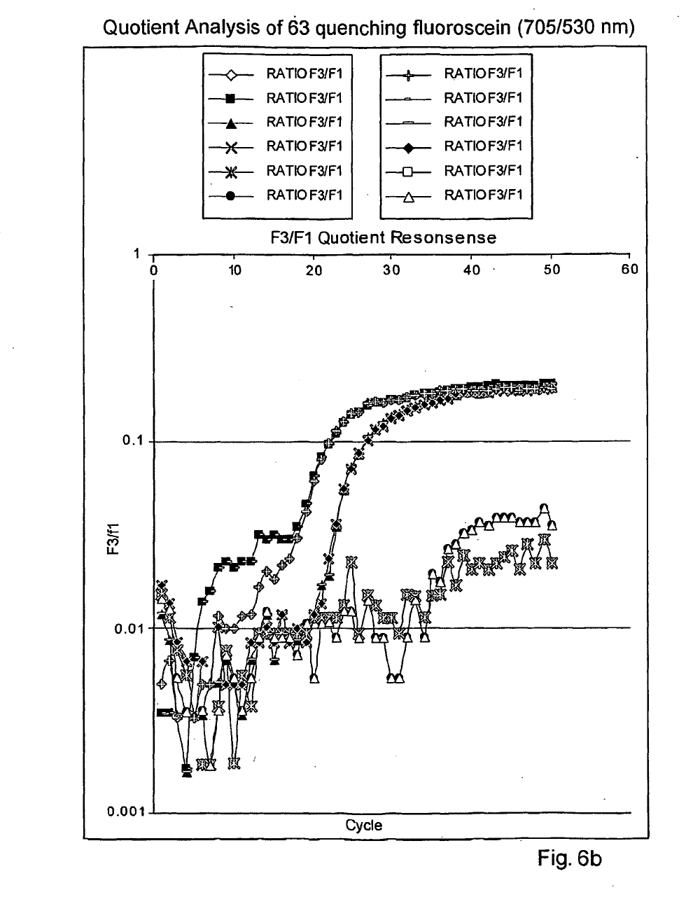

FIGS. 5-6 show the reciprocal plot of fluorescence in relation to the SYTO 63/fluoroscein experiments;

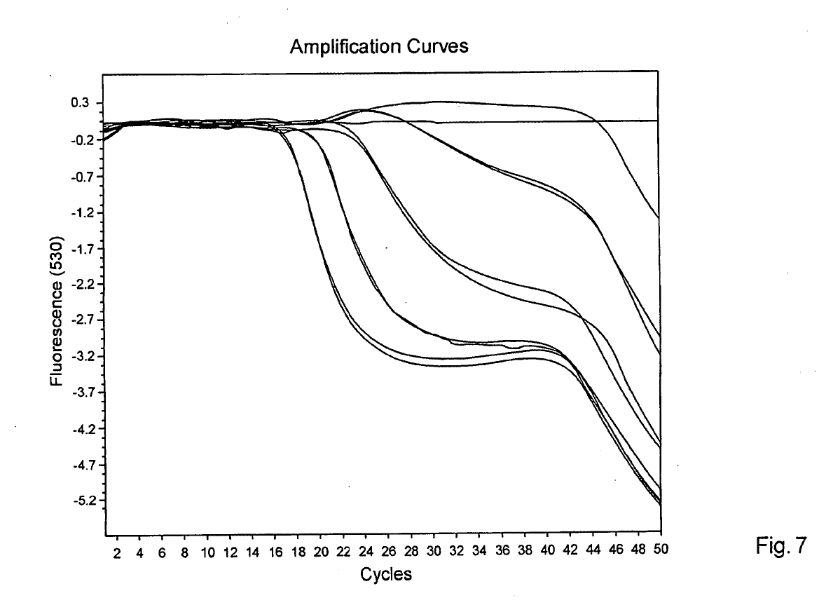

FIG. 7 shows the emission signal from a fluorophore (FAM) attached to an oligonucleotide probe for the target nucleic acid used in the assay;

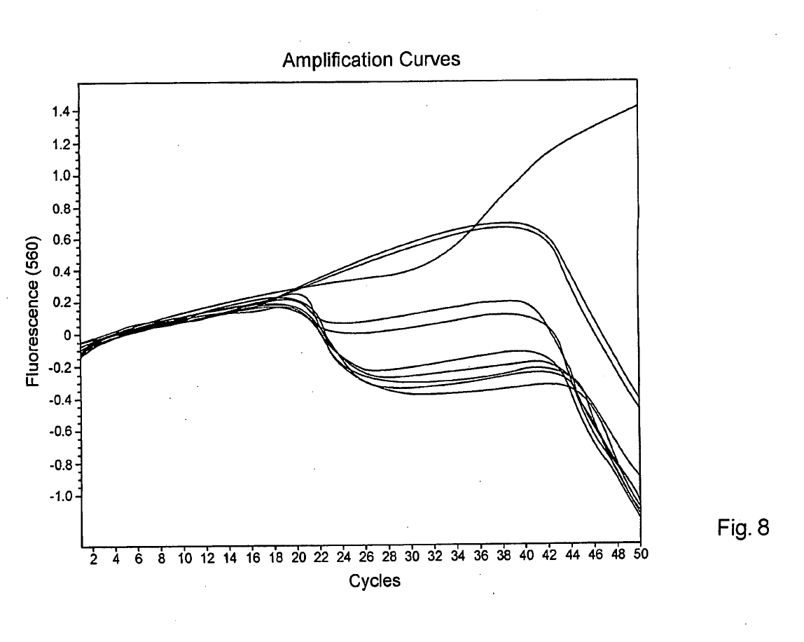

FIG. 8 shows the emission signal from a fluorophore (JOE) on an internal control used in the same assay; and

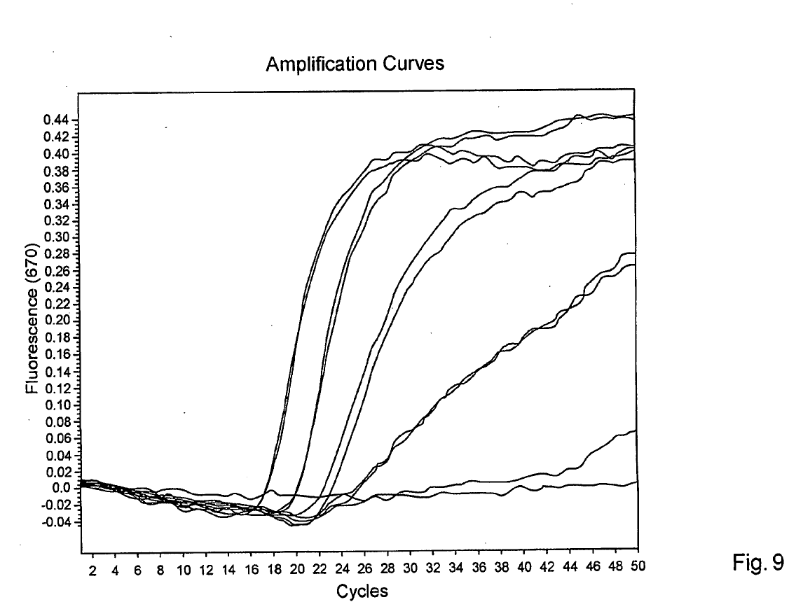

FIG. 9 shows the emission signal from SYTO 63 when used in the context of a ResonSense™ assay as shown in FIGS. 7 and 8.

### EXAMPLE 1

The applicants have carried out experiments utilising a fluorescein labelled probe and a range of nucleic acid stains, from the SYTO® red family of nucleic acid stains. It has been found that these dyes can be added into PCR without inhibition and can be added at high concentrations. They are available in a large range of wavelengths such that they can be combined with a number of fluorophores on probes.

PCRs in a ResonSense® format for a Bacillus globii gene sequence were carried out using the following experimental protocol.

An aqueous PCR mix formulation was prepared and comprised the following components:
Tris pH 8.8 at 50 mM
Bovine Serum Albumin (BSA) 250 ng/μl
Magnesium chloride 3 mM
dUTP Nucleotides at 200 μM
Taq Polymerase at 0.04 units/μl
antiTaq antibody at 0.04 units/μl
Forward Primer 1 μM
Reverse Primer at 1 μM
SYTO®* dye at 5 μM
Fluorescein labelled probe 0.2 μM

Negative control reactions without either the fluorescein labelled probe or the SYTO® dye were also run.

SYTO dyes tested included SYTO® 60, SYTO® 61 and SYTO® 63 are The reaction mixtures were then spun down and run on the Roche Lightcycler on the following cycle programme:

### Denaturation

95° C. for 5 minutes

### Cycle x 50

95° C. for 5 seconds
55° C. for 20 seconds
74° C. for 5 seconds
Melt analysis x 1
55° C. for 15 seconds

Slow ramp to 95° C. at 0.1° C./second. Fluorescence collected throughout this step.

Dyes tested successfully quenched the fluorescein signal throughout the procedure.

The results of these experiments is shown in FIGS. 1-6. in FIGS. 1-4, the F1 channel shows the drop in fluorescein with the amplification of 2 DNA dilutions. The F3 channel shows a drop in the red emission of the intercalator.

The graphs (FIGS. 5 and 6) show the reciprocal plot of fluorescence in relation to the SYTO 63/fluorescein experiments. The ratio of SYTO 63/Fluorescein is also given. However, because there is no spectral overlap from the Syto 63 (F3 channel) to the Fluorescein F1 channel there is no need to either monitor, or spectrally de-convolute these energies in order to analyse the result.

### EXAMPLE 2

PCRs in a ResonSense® format for a Foot and Mouth Disease Virus (FMDV) gene sequence were carried out using the following experimental protocol. Primers and a probe for the target were designed using conventional methods. The probe was labelled with a FAM molecule. An internal control nucleic acid was added to the sample, together with a JOE labelled probe therefore.

The following protocol was used.

| | Stock | | Final | | Volume |
|---|---|---|---|---|---|
| | Concen- | | concen- | | per |
| Reagent | tration | | tration | | 20 μl |
| PCR grade water | — | | — | | 0.35 |
| Tris buffer pH 8.8 | 500 | mM | 50 | mM | 2 |
| BSA | 20 | mg/ml | 0.25 | μg/μl | 0.25 |
| MgCl₂ | 100 | mM | 3 | mM | 0.6 |
| DUTPs | 2 | mM | 0.2 | mM | 2 |
| FMDV forward | 10 | μM | 1 | μM | 2 |
| PRIMER | | | | | |
| FMDV reverse | 10 | μM | 1 | μM | 2 |
| PRIMER | | | | | |
| FMDV PROBE | 2 | μM | 0.2 | μM | 2 |
| (FAM-labelled) | | | | | |
| LAM160 ACC PROBE | 2 | μM | 0.2 | μM | 2 |
| (JOE-labelled) | | | | | |
| Syto63 | 50 | μM | 5 | μM | 2 |
| Taq Polymerase | 5 U/ul | | 0.08 U/ul | | 0.32 |
| Antibody | Taq Pol | | Taq Pol | | |
| | equivalent | | equivalent | | |
| Taq Polymerase | 5 | U/μl | 0.04 | U/μl | 0.16 |
| Superscript III | 5 | U/ul | 0.08 | U/ul | 0.32 |
| Reverse | | | | | |
| Transcriptase | | | | | |

| | | | |
|---|---|---|---|
| FMDV RNA template | — | — | 2 |
| FMDV-λ Internal | — | — | 2 |
| Control RNA | | | |

### Thermal Parameters

| | | |
|---|---|---|
| 48° C. 5 min 20° C./sec | | |
| | {close oversize brace} | REVERSE TRANSCRIPTION |
| 95° C. 2 min 20° C./sec | | DENATURATION |
| 95° C. 5 sec 20° C./sec | | |
| | {close oversize brace} | PCR AMPLIFICATION ×50 cycles, |
| 55° C. 20 sec 20° C./sec | | fluorescence acquisition |
| 74° C. 5 sec 20° C./sec | | at end of 55° C. |
| | | annealing phase |

During the reaction, the FAM was excited by the blue LED of the LC 2.0 instrument.

### Read Parameters

Using FAM-JOE-Syto63 colour compensation:

FMDV-specific template 530 nm (FAM 521 nm)
FMDV-λ competitive Internal Control 560 nm (JOE 548 nm)

The results over a range of dilutions from 10⁻³-10⁻⁶ of the samples are shown in FIG. 7-9 respectively.

It is clear from FIG. 7 that as the amplicon accumulates during the PCR, the FAM label on the probe becomes quenched as the SYTO 63 accepts energy from it. The FAM signal is clearly seen to be dropping with increasing cycle number and different amounts of starting template, indicating that this is a reliable signalling system for monitoring amplification.

A similar signal is seen with the JOE signal from the internal control sequence (FIG. 8). The FAM and JOE signals were resolved using the colour compensation algorithm of the LightCycler 2.0 software.

In contract, as is shown in FIG. 9, emission from the FAM sequence, measured at 670 nm increases with increasing cycle number and different amounts of starting template. It is therefore absorbing energy from the FAM and JOE signals.

1. A method of using a red nucleic acid stain in the detection of nucleic acids in a polymerase chain reaction (PCR).

2. The method according to claim 1 wherein the PCR is a real-time PCR.

3. The method according to claim 1 wherein the nucleic acid stain emits fluorescence at wavelengths in excess of 600 nm.

4. The method according to claim 3 wherein the nucleic acid stain emits fluorescence at wavelengths of from 610-690 nm.

5. The method according to claim 1 wherein the dye is a SYTO® Red Fluorescent nucleic acid stain.

6. The method according to claim 5 wherein the SYTO® dye is selected from SYTO® 17, SYTO® 59, SYTO® 60, SYTO® 61, SYTO® 62, SYTO® 63 and SYTO® 64.

7. The method according to claim 1 wherein the nucleic acid stain is a red stain which is of formula (IIA) 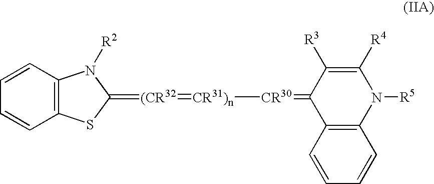
where n is 0, 1 or 2;
R² is hydrogen, an alkyl group having 1-6 carbons that is optionally substituted by sulphonate, carboxy, or amino;
R³ and R⁴ are independently H; an alkyl that is saturated or unsaturated, linear or branched, having 1-6 carbons; or a halogen; or a cyclic group (selected from an aryl, heteroaryl, or cycloalkyl having 3-10 carbons any of which may be optionally substituted by halogen, amino, alkyl, perfluoroalkyl, alkylamino, dialkylamino, alkoxy or carboxyalkyl, wherein each alkyl group has 1-6 carbons, or by a TAIL moiety); or —OR⁸, —SR⁸, —(NR⁸R⁹); or TAIL; where R⁸ and R⁹, which can be the same or different, are independently alkyl groups having 1-6 carbons; or 1-2 alicyclic or aromatic rings; or R⁸ and R⁹ taken in combination are —(CH₂)₂—V—(CH₂)₂— where V is a single bond, -0-, —CH₂—, or —NR¹⁰—, where R¹⁰ is H or an alkyl having 1-6 carbons;
R⁵ is a heteroaryl group;
R³⁰, R³¹, and R³² are independently H, alkyl having 1-6 carbons, cycloalkyl having 3-10 carbons, aryl, or heteroaryl; and TAIL is a heteroatom-containing moiety.

8. The method according to claim 7 wherein R² is an alkyl group having from 1 to 6 carbon atoms.

9. The method according to claim 7 wherein n is 0.

10. The method according to claim 7 wherein R⁵ pyridyl.

11. The method according to claim 7 wherein R³ is hydrogen and R⁴ is a TAIL.

12. The method according to claim 11 wherein the TAIL group is a group of
LINK-SPACER-CAP
where LINK is a group NR²⁰ where R²⁰ is selected from hydrogen, C₁₋₈alkyl group or a group
-SPACER′-CAP′
where SPACER′ and CAP′ are groups as defined below for SPACER and CAP respectively,
SPACER is 1-6 carbon atoms in a linear or branched saturated chain, and CAP is a group OR²¹, —SR²¹, —NR²¹R²², or —N⁺R²¹R²²R²³.PSI⁻ where R²¹, R²², and R²³ are independently H, or an optionally substituted linear or branched alkyl or cycloalkyl group having 1-8 carbons and PSI⁻ is a counterion.

13. The method use according to claim 11 wherein the TAIL group is a group of sub-formula (i), (ii), or (iii)

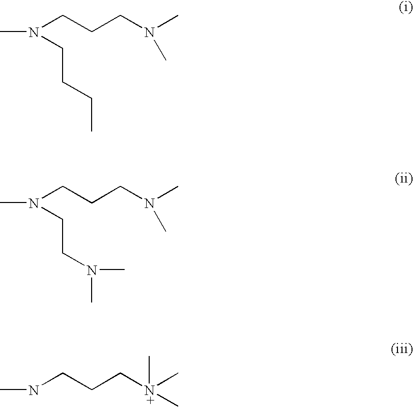

14. The method according to claim 7 wherein the compound of formula (IIA) is a compound of formula (III)

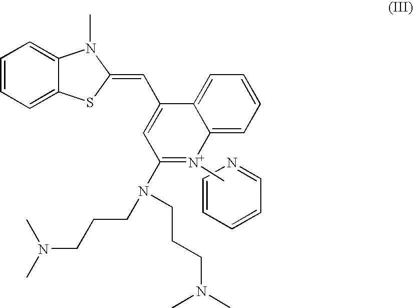

15. A method for detecting a nucleic acid sequence in a biological sample during amplification comprising the steps of:
adding a thermostable polymerase and primers configured for amplification of the target nucleic acid sequence to the biological sample;
amplifying the target nucleic acid sequence by the polymerase chain reaction in the presence of a red fluorescent nucleic acid stain and optionally additional signalling fluorophores;
illuminating the biological sample with light at a wavelength absorbed by either the nucleic acid stain or the optional additional fluorophore; and
detecting a fluorescent emission from the sample related to the presence or amount of amplified target nucleic acid sequence in the sample.

16. A method for detecting the presence of a target nucleic acid sequence in a sample, said method comprising:
(a) adding to the sample, a thermostable polymerase, primers configured for amplification of the target nucleic acid sequence, a DNA duplex binding agent, and a probe specific for said target sequence, said probe comprising a reactive molecule able to absorb fluorescence from or donate fluorescent energy to said DNA duplex binding agent, wherein one of said reactive molecule or said DNA duplex binding agent is a red fluorescent nucleic acid stain, and the other is a fluorophore, such as fluorescein or derivatives thereof,
(b) subjecting the thus formed mixture to an amplification reaction in which target nucleic acid is amplified,
(c) subjecting said sample to conditions under which the said probe hybridises to the target sequence, and
(d) monitoring fluorescence from said sample.

17. A method according to claim 16 wherein the sample is illuminated by light of a wavelength absorbed by the reactive molecule and the emission signal from the reactive molecule is monitored.

18. A method according to claim 16 wherein the nucleic acid stain is used as the DNA duplex binding agent.

19. A method according to claim 16 wherein the amplification reaction is a polymerase chain reaction.

20. A method for detecting nucleic acid amplification comprising:
performing nucleic acid amplification on a target polynucleotide in the presence of (a) a nucleic acid polymerase (b) at least one primer capable of hybridising to said target polynucleotide, (c) an oligonucleotide probe which is capable of binding to said target polynucleotide sequence and which contains a fluorescent label and (d) a red fluorescent nucleic acid stain, which is capable of absorbing fluorescent energy from the said fluorescent label; and monitoring changes in fluorescence during the amplification reaction.

21. A method according to claim 20 wherein the sample is illuminated by light of a wavelength absorbed by the fluorescent label and the emission signal from the fluorescent molecule is monitored.

22. A method according to claim 20 wherein data is taken from a sample reaction and the following equations are applied to every datapoint:
y=1/x
z=y−MIN
where x is the datapoint from the PCR machine, such as a LightCyler, Z is the baseline adjusted datapoint and MIN is the minimum value for y over the entire dataset.

23. A method for determining a characteristic of a sequence, said method comprising;
a) adding to a sample suspected of containing said sequence, a fluorescently labelled probe specific for said target sequence and a DNA duplex binding agent able to absorb fluorescence from a fluorescent label on the probe, wherein one of the label on the probe or the DNA duplex binding agent is a red fluorescent nucleic acid stain,
(b) subjecting said sample to a variable reaction condition, during which the said probe hybridises to the target sequence, and
(c) monitoring fluorescence from said sample and determining a particular reaction condition, characteristic of said sequence, at which fluorescence changes as a result of the hybridisation of the probe to the sample or destabilisation of the duplex formed between the probe and the target nucleic acid sequence.

24. A kit for use in the detection of a nucleic acid, the detection of the progress of nucleic acid amplification or for determining a characteristic of a nucleic acid sequence, said kit comprising a red fluorescent nucleic acid stain.

25. A kit according to claim 24 which further comprises one or more reagents used in a nucleic acid amplification reaction.

26. A kit according to claim 25 wherein the nucleic acid amplification reaction is a polymerase chain reaction.

27. A kit according to claim 24 which further comprises a fluorescently labelled probe specific for a nucleic acid target sequence, wherein the nucleic acid stain absorbs fluorescence from a fluorescent label on the probe.
